(19) United States
(12) Patent Application Publication
(10) Pub. No.: US 2025/0000856 A1
(43) Pub. Date: Jan. 2, 2025

(54) CRYSTALLINE FORMS OF N-((1R,3S)-3-(4-ACETYLPIPERAZIN-1-YL)CYCLOHEXYL)-4-FLUORO-7-METHYL-1H-INDOLE-2-CARBOXAMIDE

(71) Applicants: William H. CONNORS, Lynnfield, MA (US); Stephen W. RASO, Wilmington (CN); Epizyme, Inc., Cambridge, MA (US)

(72) Inventors: William H. Connors, Lynnfield, MA (US); Stephen W. Raso, Wilmington, MA (US); Meiting Shi, Beijing (CN); Ruiping Wang, Beijing (CN)

(21) Appl. No.: 18/706,071

(22) PCT Filed: Oct. 31, 2022
(86) PCT No.: PCT/US2022/078962

§ 371 Date: Apr. 30, 2024

Related U.S. Application Data
(60) Provisional application No. 63/280,972, filed on Nov. 18, 2021.

(30) Foreign Application Priority Data
Nov. 1, 2021 (WO) PCT/CN2021/127945

(51) Int. Cl. A61K 31/496; A61K 9/20; A61K 9/28; A61K 45/06; C07D 209/42

(52) U.S. Cl. CPC A61K 31/496; A61K 9/2013; A61K 9/2018; A61K 9/2054; A61K 9/2095; A61K 9/2866; A61K 9/2893; A61K 45/06; C07D 209/42

(57) ABSTRACT

This disclosure provides crystalline forms of N-((1R,3S)-3-(4-acetylpiperazin-1-yl)cyclohexyl)-4-fluoro-7-methyl-1H-indole-2-carboxamide. N ((1R,3S)-3-(4-acetylpiperazin-1-yl)cyclohexyl)-4-fluoro-7-methyl-1H-indole-2-carboxamide hydrochloride, and N-((1R,3S)-3-(4-acetylpiperazin-1-yl)cyclohexyl)-4-fluoro-7-methyl-1H-indole-2-carboxamide hydrobromide, pharmaceutical compositions comprising these crystalline forms, methods of making these crystalline forms, and methods of treating a disease, condition, or disorder in a subject comprising administering these crystalline forms to the subject.

### BACKGROUND OF THE INVENTION

### Field of Invention

This disclosure provides crystalline forms of N-((1R,3S)-3-(4-acetylpiperazin-1-yl)cyclohexyl)-4-fluoro-7-methyl-1H-indole-2-carboxamide (“Compound 1”), N-((1R,3S)-3-(4-acetylpiperazin-1-yl)cyclohexyl)-4-fluoro-7-methyl-1H-indole-2-carboxamide hydrochloride (“Compound 1 HCl”), and N-((1R,3S)-3-(4-acetylpiperazin-1-yl)cyclohexyl)-4-fluoro-7-methyl-1H-indole-2-carboxamide hydrobromide (“Compound 1 HBr”); pharmaceutical compositions comprising crystalline forms of Compound 1, Compound 1 HCl, and Compound 1 HBr; methods of making crystalline forms of Compound 1, Compound 1 HCl, and Compound 1 HBr; and methods of treating a disease, condition, or disorder in a subject comprising administering crystalline forms of Compound 1, Compound 1 HCl, and Compound 1 HBr to the subject.

### Background

N-((1R,3S)-3-(4-acetylpiperazin-1-yl)cyclohexyl)-4-fluoro-7-methyl-1H-indole-2-carboxamide (“Compound 1”) is a small molecule inhibitor of the enzymatic activity of histone methyltransferase (HMT) Su(var)3-9, Enhancer-of-zeste, Trithorax domain containing 2 (SETD2), also known as KMT3A. This compound and its method of synthesis is disclosed in WO 2020/037079.

There exists a need for solid forms of Compound 1 for use in treating cancer and other diseases, disorders, and conditions in a subject.

### BRIEF SUMMARY OF THE INVENTION

In one aspect, the present disclosure provides crystalline forms of Compound 1, crystalline forms of Compound 1 HCl, and crystalline forms of Compound 1 HBr.

In another aspect, the present disclosure provides pharmaceutical compositions comprising crystalline forms of Compound 1, crystalline forms of Compound 1 HCl, or crystalline forms of Compound 1 HBr and one or more pharmaceutically acceptable excipients.

In another aspect, the present disclosure provides methods of making crystalline forms of crystalline forms of Compound 1, crystalline forms of Compound 1 HCl, or crystalline forms of Compound 1 HBr.

In another aspect, the present disclosure provides a method of administering crystalline forms of Compound 1, crystalline forms of Compound 1 HCl, or crystalline forms of Compound 1 HBr to treat a disease, disorder, or condition, e.g., cancer, in a subject in a subject in need thereof.

In another aspect, the present disclosure provides crystalline forms of Compound 1, crystalline forms of Compound 1 HCl, or crystalline forms of Compound 1 HBr, or a composition thereof, for use in treating a disease, disorder, or condition, e.g., cancer, in a subject.

In another aspect, the present disclosure provides crystalline forms of Compound 1, crystalline forms of Compound 1 HCl, or crystalline forms of Compound 1 HBr for use in the manufacture of a medicament to treat a disease, disorder, or condition, e.g., cancer, in a subject in a subject.

In another aspect, the present disclosure provides a kit comprising crystalline forms of Compound 1, crystalline forms of Compound 1 HCl, or crystalline forms of Compound 1 HBr.

In another aspect, the present disclosure provides a method of making a pharmaceutical composition comprising crystalline forms of Compound 1, crystalline forms of Compound 1 HCl, or crystalline forms of Compound 1 HBr and one or more pharmaceutically acceptable excipients.

### BRIEF DESCRIPTION OF DRAWINGS

FIG. 1 is a XRPD diffractogram of Free Base Form I.

FIG. 2 is a XRPD diffractogram of Free Base Form II.

FIG. 3 is a FTIR spectrum of Free Base Form II.

FIG. 4 is a DSC and TGA thermogram of Free Base Form II.

FIG. 5 is a XRPD diffractogram of Free Base Form III.

FIG. 6 is a DSC and TGA thermogram of Free Base Form III.

FIG. 7 is a XRPD diffractogram of Free Base Form IV.

FIG. 8 is a DSC and TGA thermogram of Free Base Form IV.

FIG. 9 is a XRPD diffractogram of HCl Form I.

FIG. 10 is a DSC and TGA thermogram of HCl Form I.

FIG. 11 is a XRPD diffractogram of HCl Form II.

FIG. 12 is a DSC and TGA thermogram of HCl Form II.

FIG. 13 is a XRPD diffractogram of HBr Form I.

FIG. 14 is a DSC and TGA thermogram of HBr Form I.

FIG. 15 is a line graph showing the mean plasma concentration versus time profiles of Compound 1 in male beagle dogs following a single oral dose of Free Base Form I and Free Base Form II. Data are expressed as Mean±SD with n=3 for each group.

FIG. 16 is a process flow diagram for preparing tablets comprising Free Base Form II.

### DETAILED DESCRIPTION OF THE INVENTION

### I. Crystalline Forms of the Disclosure

In one embodiment, the present disclosure provides crystalline forms of Compound 1. These are referred to as the “free base” forms.

### Free Base Form I

In another embodiment, the crystalline form of Compound 1 is characterized as having a powder x-ray diffraction pattern with peaks at 7.0, 9.8, 14.1, 14.9, 15.2, 17.5, 18.1, 19.2, 20.0, 20.7, 22.5, and 23.9 degrees 2Θ using Cu Kα radiation, wherein the 20 values are ±0.2 degrees 2Θ. This crystalline form is referred to as “Free Base Form I.” Free Base Form I is a mixture of Free Base Form II and Free Base Form IV. See below.

In another embodiment, Free Base Form I is characterized as having a powder x-ray diffraction pattern with peaks at 7.0, 14.0, 18.0, 20.0, and 20.7 degrees 2Θ using Cu Kα radiation, wherein the 2Θ values are ±0.2 degrees 2Θ.

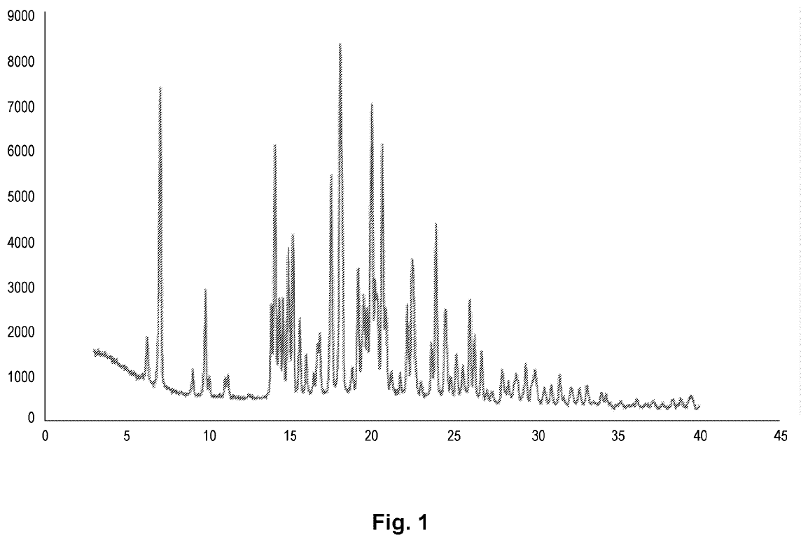

In another embodiment, Free Base Form I is characterized as having a XRPD diffractogram that is essentially the same as the one depicted in FIG. 1 using Cu Kα radiation.

In another embodiment, Free Base Form I is characterized as having thermal events with an onset temperature of about 41.9° C. and a peak temperature of about 62.6° C.; an onset temperature of about 70.5° C. and a peak temperature of about 80.0° C.; an onset temperature of about 132.9° C. and a peak temperature of about 144.1; and an onset temperature of about 214.5° C. and a peak temperature of about 220.0° C. based on differential scanning calorimetry (DSC).

### Free Base Form II

In another embodiment, the crystalline form of Compound 1 is characterized as having a powder x-ray diffraction pattern with the peaks listed in Table A using Cu Kα radiation, wherein the 2Θ values are ±0.2 degrees 2Θ. This crystalline form is referred to as “Free Base Form II.” Free Base Form II is anhydrous.

In another embodiment, Free Base Form II is characterized as having a powder x-ray diffraction pattern with the d-spacings listed in Table A using Cu Kα radiation.

In another embodiment, Free Base Form II is characterized as having a powder x-ray diffraction pattern with peaks at 7.0, 9.8, 11.1, 14.0, 15.2, 15.5, 16.8, 17.5, 18.0, 19.6, 20.2, 20.8, 21.1, 22.1, 22.5, 23.9, 24.4, 24.8, 25.1, 26.0, 28.3, 29.4, and 30.0 degrees 2Θ using Cu Kα radiation, wherein the 2Θ values are ±0.2 degrees 2Θ.

In another embodiment, Free Base Form II is characterized as having a powder x-ray diffraction pattern with at least three peaks at 7.0, 9.8, 10.0, 11.0, 11.2, 14.0, 14.3, 15.1, 15.5, 15.9, 16.2, 16.8, 17.5, 18.0, 19.4, 19.6, 20.2, 20.8, 21.1, 22.1, 22.4, 23.8, 24.4, 24.8, 25.1, 25.7, 25.9, 26.2, 27.2, 27.9, 28.3, 28.6, 29.4, 30.0, 30.4, 31.0, 31.4, 32.1, 32.7, 33.1, 33.4, 34.0, 34.3, 34.9, 35.1, 36.1, 36.5, 37.1, 37.7, 38.3, 38.6, 38.8, and/or 39.4 degrees 2Θ using Cu Kα radiation, wherein the 2Θ values are ±0.2 degrees 2Θ.

In another embodiment, Free Base Form II is characterized as having a powder x-ray diffraction pattern with at least three peaks at 7.0, 9.8, 11.1, 14.0, 15.2, 15.5, 16.8, 17.5, 18.0, 19.6, 20.2, 20.8, 21.1, 22.1, 22.5, 23.9, 24.4, 24.8, 25.1, 26.0, 28.3, 29.4, and/or 30.0 degrees 2Θ using Cu Kα radiation, wherein the 2Θ values are ±0.2 degrees 2Θ.

In another embodiment, Free Base Form II is characterized as having a powder x-ray diffraction pattern with at least four peaks at 7.0, 9.8, 10.0, 11.0, 11.2, 14.0, 14.3, 15.1, 15.5, 15.9, 16.2, 16.8, 17.5, 18.0, 19.4, 19.6, 20.2, 20.8, 21.1, 22.1, 22.4, 23.8, 24.4, 24.8, 25.1, 25.7, 25.9, 26.2, 27.2, 27.9, 28.3, 28.6, 29.4, 30.0, 30.4, 31.0, 31.4, 32.1, 32.7, 33.1, 33.4, 34.0, 34.3, 34.9, 35.1, 36.1, 36.5, 37.1, 37.7, 38.3, 38.6, 38.8, and/or 39.4 degrees 2Θ using Cu Kα radiation, wherein the 2Θ values are ±0.2 degrees 2Θ.

In another embodiment, Free Base Form II is characterized as having a powder x-ray diffraction pattern with at least four peaks at 7.0, 9.8, 11.1, 14.0, 15.2, 15.5, 16.8, 17.5, 18.0, 19.6, 20.2, 20.8, 21.1, 22.1, 22.5, 23.9, 24.4, 24.8, 25.1, 26.0, 28.3, 29.4, and/or 30.0 degrees 2Θ using Cu Kα radiation, wherein the 2Θ values are ±0.2 degrees 2Θ.

In another embodiment, Free Base Form II is characterized as having a powder x-ray diffraction pattern with at least five peaks at 7.0, 9.8, 10.0, 11.0, 11.2, 14.0, 14.3, 15.1, 15.5, 15.9, 16.2, 16.8, 17.5, 18.0, 19.4, 19.6, 20.2, 20.8, 21.1, 22.1, 22.4, 23.8, 24.4, 24.8, 25.1, 25.7, 25.9, 26.2, 27.2, 27.9, 28.3, 28.6, 29.4, 30.0, 30.4, 31.0, 31.4, 32.1, 32.7, 33.1, 33.4, 34.0, 34.3, 34.9, 35.1, 36.1, 36.5, 37.1, 37.7, 38.3, 38.6, 38.8, and/or 39.4 degrees 2Θ using Cu Kα radiation, wherein the 2Θ values are ±0.2 degrees 2Θ.

In another embodiment, Free Base Form II is characterized as having a powder x-ray diffraction pattern with at least five peaks at 7.0, 9.8, 11.1, 14.0, 15.2, 15.5, 16.8, 17.5, 18.0, 19.6, 20.2, 20.8, 21.1, 22.1, 22.5, 23.9, 24.4, 24.8, 25.1, 26.0, 28.3, 29.4, and/or 30.0 degrees 2Θ using Cu Kα radiation, wherein the 2Θ values are ±0.2 degrees 2Θ.

In another embodiment, Free Base Form II is characterized as having a powder x-ray diffraction pattern with peaks at 7.0, 14.0, 18.0, and 20.2 degrees 2Θ using Cu Kα radiation wherein the 2Θ values are ±0.2 degrees 2Θ.

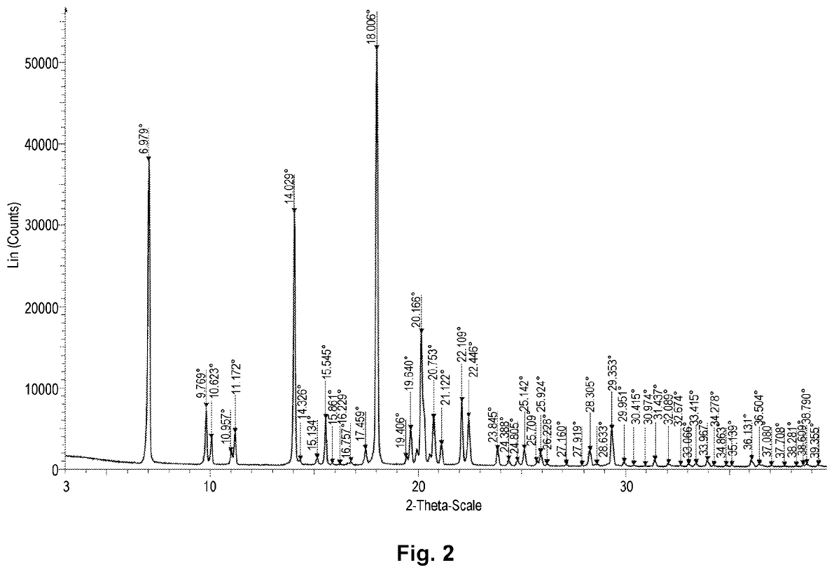

In another embodiment, Free Base Form II is characterized as having a XRPD diffractogram that is essentially the same as the one depicted in FIG. 2 using Cu Kα radiation.

In another embodiment, Free Base Form II is characterized as having infrared (IR) spectrum with stretches at 3292, 2934, 2860, 1628, 1528, 1508, 1449, 1248, 791, 777, and 745 cm⁻¹, wherein the cm 1 values are ±4 cm⁻¹.

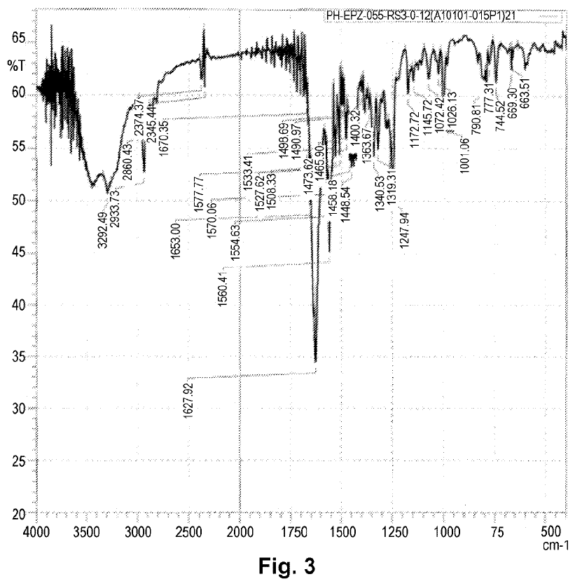

In another embodiment, Free Base Form II is characterized as having an IR spectrum that is essentially the same as the one depicted in FIG. 3.

In another embodiment, Free Base Form II is characterized as having a melting point with an onset temperature of about 224.3° C. and a peak temperature of about 225.7° C. based on differential scanning calorimetry (DSC).

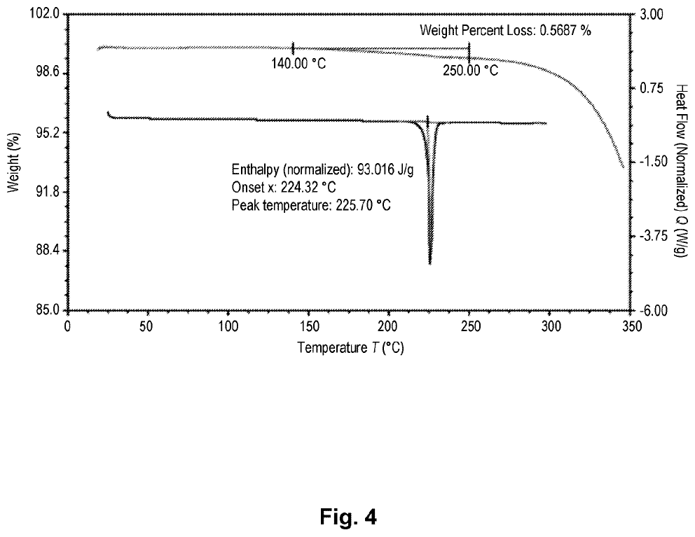

In another embodiment, Free Base Form II is characterized as having a DSC thermogram that is essentially the same as the one depicted in FIG. 4

### Free Base Form III

In another embodiment, the crystalline form of Compound 1 is characterized as having a powder x-ray diffraction pattern with the peaks listed in Table B using Cu Kα radiation, wherein the 2Θ values are ±0.2 degrees 2Θ. This crystalline form is referred to as “Free Base Form III.” Free Base Form III is the dehydrated form of Free Base Form IV. See below.

In another embodiment, Free Base Form III is characterized as having a powder x-ray diffraction pattern with the d-spacings listed in Table B using Cu Kα radiation.

In another embodiment, Free Base Form III is characterized as having a powder x-ray diffraction pattern with peaks at 7.6, 9.3, 14.8, 15.4, 15.5, 17.1, 17.5, 18.0, 19.8, 22.1, 22.7, 26.0, 27.7, and 29.8 degrees 2Θ using Cu Kα radiation, wherein the 2Θ values are ±0.2 degrees 2Θ. In another embodiment, the Free Base Form III is characterized as having a powder x-ray diffraction pattern with at least three peaks at 7.6, 9.3, 14.8, 15.4, 15.5, 17.1, 17.5, 18.0, 19.8, 22.1, 22.7, 26.0, 27.7, and/or 29.8 degrees 2Θ using Cu Kα radiation, wherein the 2Θ values are ±0.2 degrees 2Θ.

In another embodiment, the Free Base Form III is characterized as having a powder x-ray diffraction pattern with at least three peaks at 7.6, 9.3, 14.8, 15.4, 15.5, 17.1, 17.5, 18.0, 19.8, 22.1, 22.7, 26.0, 27.7, and/or 29.8 degrees 2Θ using Cu Kα radiation, wherein the 2Θ values are ±0.2 degrees 2Θ.

In another embodiment, the Free Base Form III is characterized as having a powder x-ray diffraction pattern with at least four peaks at 7.6, 9.3, 14.8, 15.4, 15.5, 17.1, 17.5, 18.0, 19.8, 22.1, 22.7, 26.0, 27.7, and/or 29.8 degrees 2Θ using Cu Kα radiation, wherein the 2Θ values are ±0.2 degrees 2Θ.

In another embodiment, the Free Base Form III is characterized as having a powder x-ray diffraction pattern with at least five peaks at 7.6, 9.3, 14.8, 15.4, 15.5, 17.1, 17.5, 18.0, 19.8, 22.1, 22.7, 26.0, 27.7, and/or 29.8 degrees 2Θ using Cu Kα radiation, wherein the 2Θ values are ±0.2 degrees 2Θ.

In another embodiment, Free Base Form III is characterized as having a powder x-ray diffraction pattern with peaks at 7.6, 14.8, 18.0, and 19.8 degrees 2Θ using Cu Kα radiation, wherein the 2Θ values are ±0.2 degrees 2Θ.

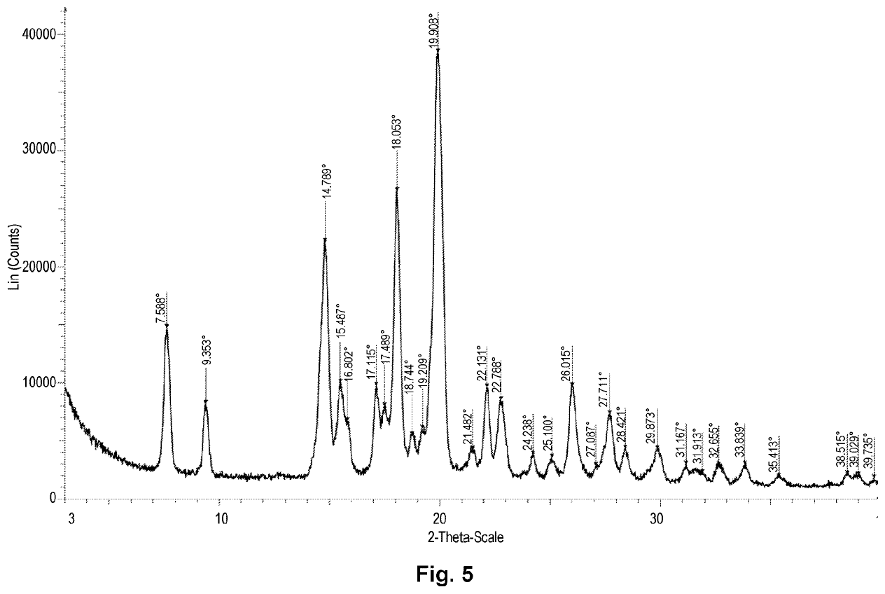

In another embodiment, Free Base Form III is characterized as having a XRPD diffractogram that is essentially the same as the one depicted in FIG. 5 using Cu Kα radiation.

In another embodiment, Free Base Form III is characterized as having thermal events with peak temperatures at about 139.0° C., 176.1° C., and 223.2° C. based on differential scanning calorimetry (DSC).

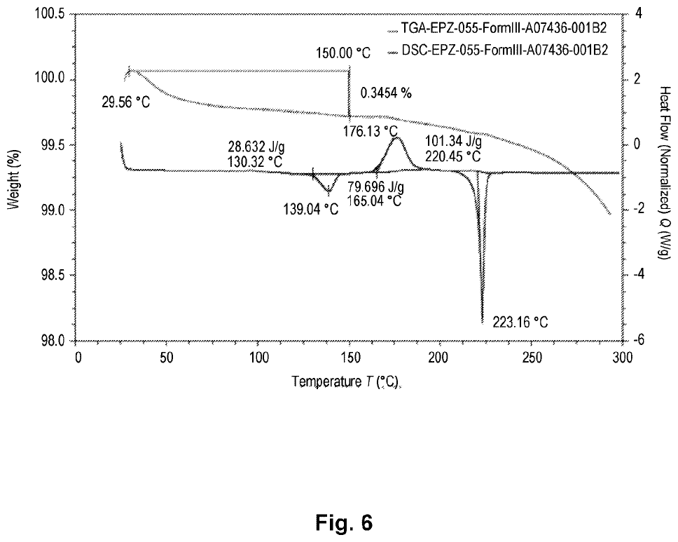

In another embodiment, Free Base Form III is characterized as having a DSC thermogram that is essentially the same as the one depicted in FIG. 6.

### Free Base Form IV

In another embodiment, the crystalline form of Compound 1 is characterized as having a powder x-ray diffraction pattern with the peaks listed in Table C using Cu Kα radiation, wherein the 2Θ values are ±0.2 degrees 2Θ. This crystalline form is referred to as “Free Base Form IV”. Free Base Form IV is a hydrate represented by the formula: Compound 1·xH₂O, wherein x is about 3.

In another embodiment, Free Base Form IV is characterized as having a powder x-ray diffraction pattern with the d-spacings listed in Table C using Cu Kα radiation.

In another embodiment, Free Base Form IV is characterized as having a powder x-ray diffraction pattern with peaks at 6.2, 7.0, 9.0, 9.8, 13.8, 14.5, 14.8, 15.9, 16.4, 18.1, 18.7, 19.1, 19.9, 20.5, 21.7, 22.4, 22.9, 23.6, 24.5, 25.5, 26.2, 26.6, 28.8, 29.8, and 30.9 degrees 2Θ using Cu Kα radiation, wherein the 2Θ values are ±0.2 degrees 2Θ. In another embodiment, Free Base Form IV is characterized as having a powder x-ray diffraction pattern with at least three peaks at 6.2, 7.0, 9.0, 9.8, 13.8, 14.5, 14.8, 15.9, 16.4, 18.1, 18.7, 19.1, 19.9, 20.5, 21.7, 22.4, 22.9, 23.6, 24.5, 25.5, 26.2, 26.6, 28.8, 29.8, and/or 30.9 degrees 2Θ using Cu Kα radiation, wherein the 2Θ values are ±0.2 degrees 2Θ.

In another embodiment, Free Base Form IV is characterized as having a powder x-ray diffraction pattern with at least four peaks at 6.2, 7.0, 9.0, 9.8, 13.8, 14.5, 14.8, 15.9, 16.4, 18.1, 18.7, 19.1, 19.9, 20.5, 21.7, 22.4, 22.9, 23.6, 24.5, 25.5, 26.2, 26.6, 28.8, 29.8, and/or 30.9 degrees 2Θ using Cu Kα radiation, wherein the 2Θ values are ±0.2 degrees 2Θ.

In another embodiment, Free Base Form IV is characterized as having a powder x-ray diffraction pattern with at least five peaks at 6.2, 7.0, 9.0, 9.8, 13.8, 14.5, 14.8, 15.9, 16.4, 18.1, 18.7, 19.1, 19.9, 20.5, 21.7, 22.4, 22.9, 23.6, 24.5, 25.5, 26.2, 26.6, 28.8, 29.8, and/or 30.9 degrees 2Θ using Cu Kα radiation, wherein the 2Θ values are ±0.2 degrees 2Θ.

In another embodiment, Free Base Form IV is characterized as having a powder x-ray diffraction pattern with peaks at 14.8, 18.1, 19.1, 19.9, and 20.5 degrees 2Θ using Cu Kα radiation, wherein the 2Θ values are ±0.2 degrees 2Θ using Cu Kα radiation, wherein the 2Θ values are ±0.2 degrees 2Θ.

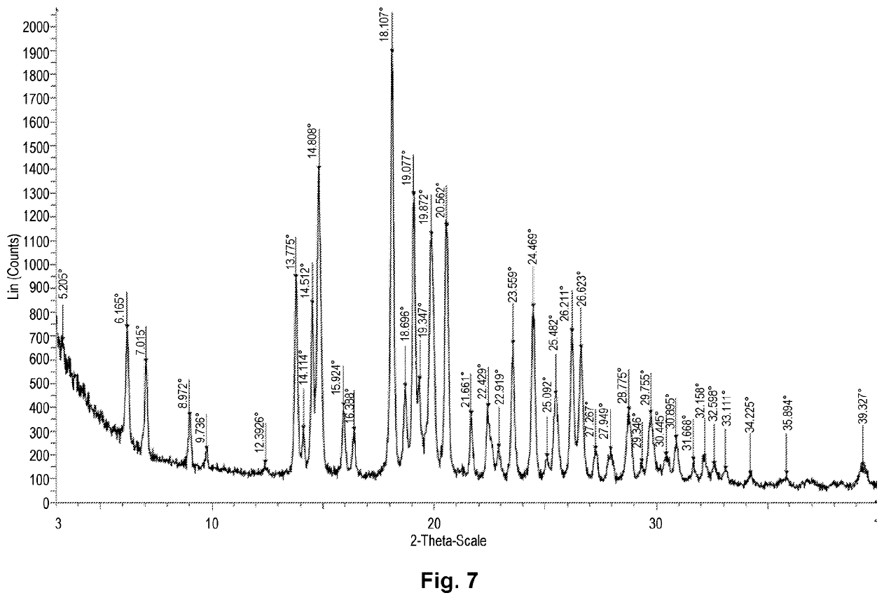

In another embodiment, Free Base Form IV is characterized as having a XRPD diffractogram that is essentially the same as the one depicted in FIG. 7 using Cu Kα radiation.

In another embodiment, Free Base Form IV is characterized as having as having thermal events with peak temperatures at about 94.7° C., 139.2° C., 166.2° C., and 222.8° C. based on differential scanning calorimetry (DSC).

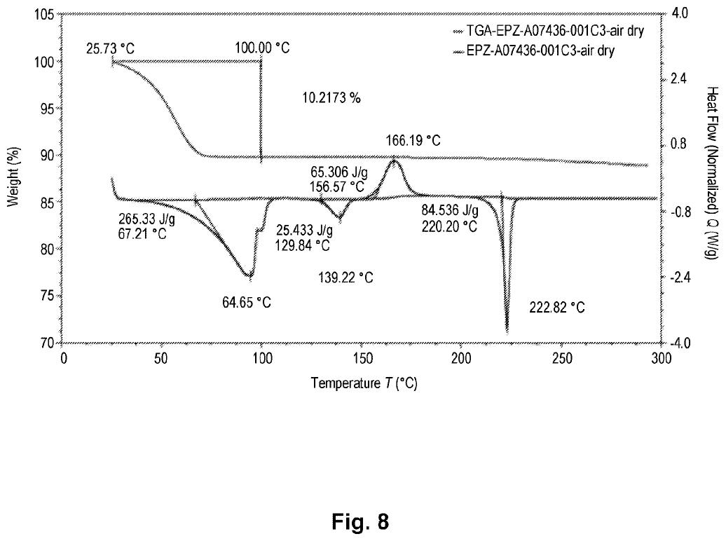

In another embodiment, Free Base Form III is characterized as having a DSC thermogram that is essentially the same as the one depicted in FIG. 8.

In another embodiment, the present disclosure provides crystalline forms of Compound 1 HCl.

### HCl Form I

In another embodiment, the crystalline form of Compound 1 is characterized as having a powder x-ray diffraction pattern with the peaks listed in Table D using Cu Kα radiation, wherein the 2Θ values are ±0.2 degrees 2Θ. This crystalline form is referred to as “HCl Form I.” HCl Form I is a hydrate represented by the formula: Compound 1 HCl·xH₂O, wherein x is about 1.

In another embodiment, HCl Form I is characterized as having a powder x-ray diffraction pattern with the d-spacings listed in Table D using Cu Kα radiation.

In another embodiment, HCl Form I is characterized as having a powder x-ray diffraction pattern with peaks at 13.6, 14.6, 14.8, 16.8, 17.6, 18.6, 20.3, 21.1, 21.6, 22.6, 24.1, 25.1, 25.4, 25.8, 26.4, 27.6, and 30.3 degrees 2Θ using Cu Kα radiation, wherein the 2Θ values are ±0.2 degrees 2Θ. In another embodiment, HCl Form I is characterized as having a powder x-ray diffraction pattern with at least three peaks at 13.6, 14.6, 14.8, 16.8, 17.6, 18.6, 20.3, 21.1, 21.6, 22.6, 24.1, 25.1, 25.4, 25.8, 26.4, 27.6, and/or 30.3 degrees 2Θ using Cu Kα radiation, wherein the 2Θ values are ±0.2 degrees 2Θ.

In another embodiment, HCl Form I is characterized as having a powder x-ray diffraction pattern with at least four peaks at 13.6, 14.6, 14.8, 16.8, 17.6, 18.6, 20.3, 21.1, 21.6, 22.6, 24.1, 25.1, 25.4, 25.8, 26.4, 27.6, and/or 30.3 degrees 2Θ using Cu Kα radiation, wherein the 2Θ values are ±0.2 degrees 2Θ.

In another embodiment, HCl Form I is characterized as having a powder x-ray diffraction pattern with at five three peaks at 13.6, 14.6, 14.8, 16.8, 17.6, 18.6, 20.3, 21.1, 21.6, 22.6, 24.1, 25.1, 25.4, 25.8, 26.4, 27.6, and/or 30.3 degrees 2Θ using Cu Kα radiation, wherein the 2Θ values are ±0.2 degrees 2Θ.

In another embodiment, HCl Form I is characterized as having a powder x-ray diffraction pattern with peaks at 13.6, 14.6, 22.6, 24.1, 25.0, and 26.4 degrees 2Θ using Cu Kα radiation, wherein the 2Θ values are ±0.2 degrees 2Θ.

In another embodiment, HCl Form I is characterized as having a powder x-ray diffraction pattern with peaks at 14.6 and 25.0 degrees 2Θ using Cu Kα radiation, wherein the 2Θ values are ±0.2 degrees 2Θ.

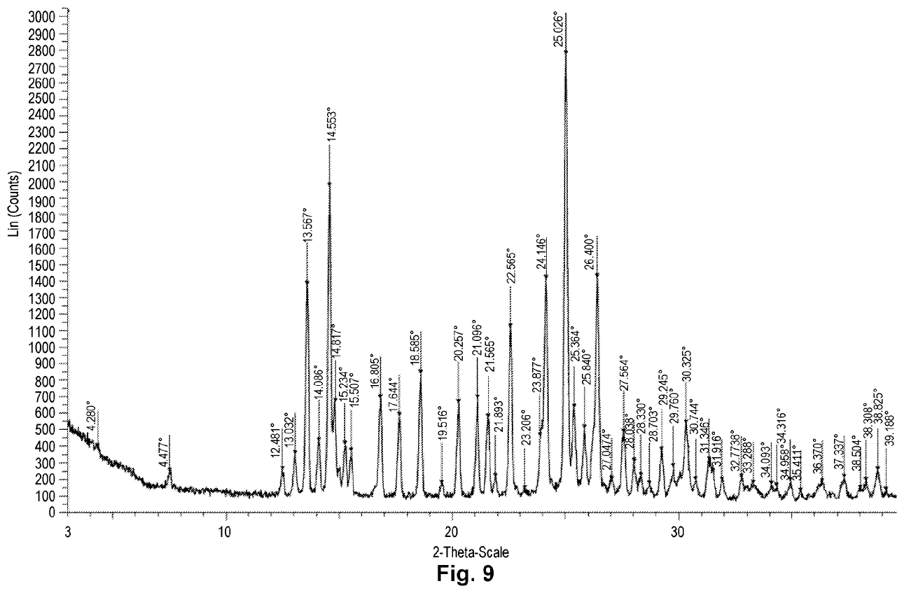

In another embodiment, HCl Form I is characterized as having a XRPD diffractogram that is essentially the same as the one depicted in FIG. 9 using Cu Kα radiation.

In another embodiment, HCl Form I is characterized as having a melting point with an onset temperature of 96.1° C. and a peak temperature of 151.4° C. based on differential scanning calorimetry (DSC).

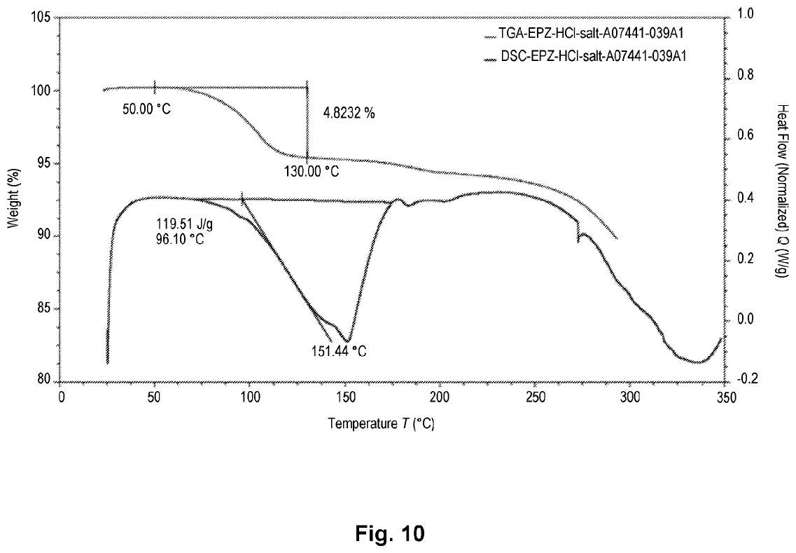

In another embodiment, HCl Form I is characterized as having a DSC thermogram that is essentially the same as the one depicted in FIG. 10.

### HCl Form II

In another embodiment, the crystalline form of Compound 1 is characterized as having a powder x-ray diffraction pattern with the peaks listed in Table E using Cu Kα radiation, wherein the 2Θ values are ±0.2 degrees 2Θ. This crystalline form is referred to as “HCl Form II.” HCl Form II is anhydrous.

In another embodiment, HCl Form II is characterized as having a powder x-ray diffraction pattern with the d-spacings listed in Table E using Cu Kα radiation.

In another embodiment, HCl Form II is characterized as having a powder x-ray diffraction pattern with peaks at 7.7, 9.3, 10.9, 13.0, 14.2, 15.2, 16.0, 16.8, 17.7, 18.7, 19.9, 21.5, 21.7, 22.6, 26.1, 27.4, 27.9, 28.6, 30.0, and 33.7 degrees 20 using Cu α radiation, wherein the 2Θ values are ±0.2 degrees 2Θ. In another embodiment, HCl Form II is characterized as having a powder x-ray diffraction pattern with at least three peaks at 7.7, 9.3, 10.9, 13.0, 14.2, 15.2, 16.0, 16.8, 17.7, 18.7, 19.9, 21.5, 21.7, 22.6, 26.1, 27.4, 27.9, 28.6, 30.0, and/or 33.7 degrees 2Θ using Cu Kα radiation, wherein the 20 values are ±0.2 degrees 2Θ.

In another embodiment, HCl Form II is characterized as having a powder x-ray diffraction pattern with at least four peaks at 7.7, 9.3, 10.9, 13.0, 14.2, 15.2, 16.0, 16.8, 17.7, 18.7, 19.9, 21.5, 21.7, 22.6, 26.1, 27.4, 27.9, 28.6, 30.0, and/or 33.7 degrees 2Θ using Cu Kα radiation, wherein the 2Θ values are ±0.2 degrees 2Θ.

In another embodiment, HCl Form II is characterized as having a powder x-ray diffraction pattern with at least five peaks at 7.7, 9.3, 10.9, 13.0, 14.2, 15.2, 16.0, 16.8, 17.7, 18.7, 19.9, 21.5, 21.7, 22.6, 26.1, 27.4, 27.9, 28.6, 30.0, and/or 33.7 degrees 2Θ using Cu Kα radiation, wherein the 2Θ values are ±0.2 degrees 2Θ.

In another embodiment, HCl Form II is characterized as having a powder x-ray diffraction pattern with peaks at 15.2, 16.0, 17.7, and 22.6 degrees 2Θ using Cu Kα radiation, wherein the 2Θ values are ±0.2 degrees 2Θ.

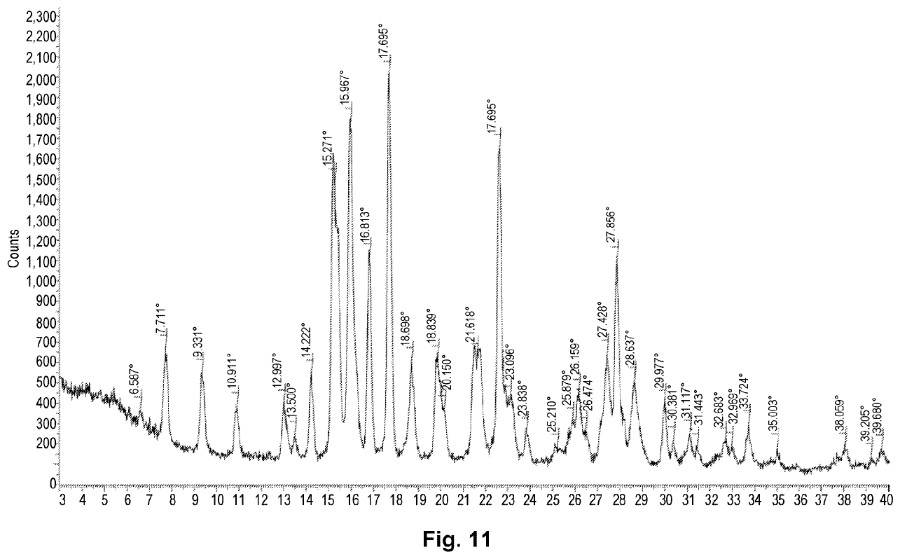

In another embodiment, HCl Form II is characterized as having a XRPD diffractogram that is essentially the same as the one depicted in FIG. 11 using Cu Kα radiation.

In another embodiment, HCl Form II is characterized as having a melting point with an onset temperature of 296.4° C. and a peak temperature of 308.3° C. based on differential scanning calorimetry (DSC).

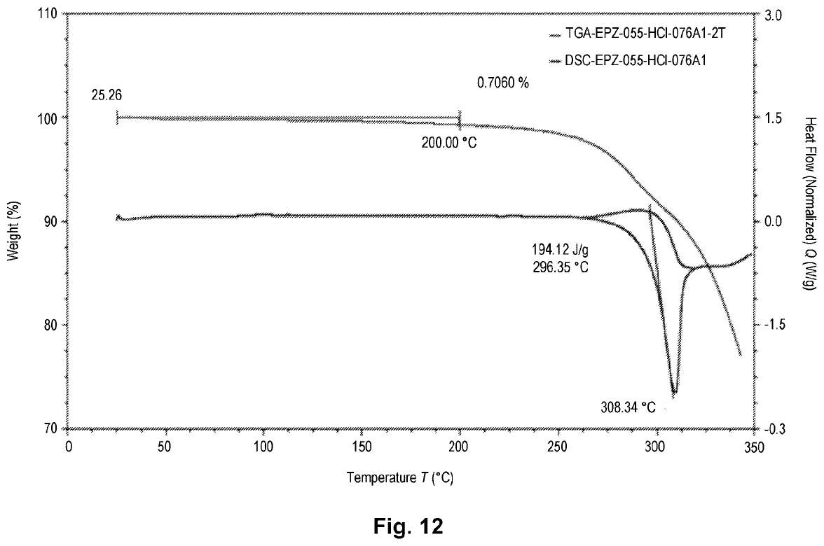

In another embodiment, HCl Form II is characterized as having a DSC thermogram that is essentially the same as the one depicted in FIG. 12.

In another embodiment, the present disclosure provides crystalline forms of Compound 1 HBr.

### HBr Form I

In another embodiment, the crystalline form of Compound 1 is characterized as having a powder x-ray diffraction pattern with the peaks listed in Table F using Cu Kα radiation, wherein the 2Θ values are ±0.2 degrees 2Θ. This crystalline form is referred to as “HBr Form I.” HBr Form I is a hydrate represented by the formula: Compound 1 HBr·xH₂O, wherein x is about 1.

In another embodiment, HBr Form I is characterized as having a powder x-ray diffraction pattern with the d-spacings listed in Table F using Cu Kα radiation.

In another embodiment, HBr Form I is characterized as having a powder x-ray diffraction pattern with peaks at 7.6, 12.3, 12.8, 13.6, 14.1, 14.5, 15.0, 15.5, 16.7, 17.7, 18.8, 19.5, 20.4, 21.2, 22.6, 24.2, 25.1, 26.0, 26.5, 27.1, 28.4, 29.3, 30.6, 31.2, 33.4, and 36.0 degrees 2Θ using Cu Kα radiation, wherein the 2Θ values are ±0.2 degrees 2Θ.

In another embodiment, HBr Form I is characterized as having a powder x-ray diffraction pattern with at least three peaks at 7.6, 12.3, 12.8, 13.6, 14.1, 14.5, 15.0, 15.5, 16.7, 17.7, 18.8, 19.5, 20.4, 21.2, 22.6, 24.2, 25.1, 26.0, 26.5, 27.1, 28.4, 29.3, 30.6, 31.2, 33.4, and/or 36.0 degrees 2Θ using Cu Kα radiation, wherein the 2Θ values are ±0.2 degrees 2Θ.

In another embodiment, HBr Form I is characterized as having a powder x-ray diffraction pattern with at least four peaks at 7.6, 12.3, 12.8, 13.6, 14.1, 14.5, 15.0, 15.5, 16.7, 17.7, 18.8, 19.5, 20.4, 21.2, 22.6, 24.2, 25.1, 26.0, 26.5, 27.1, 28.4, 29.3, 30.6, 31.2, 33.4, and/or 36.0 degrees 2Θ using Cu Kα radiation, wherein the 2Θ values are ±0.2 degrees 2Θ.

In another embodiment, HBr Form I is characterized as having a powder x-ray diffraction pattern with at least five peaks at 7.6, 12.3, 12.8, 13.6, 14.1, 14.5, 15.0, 15.5, 16.7, 17.7, 18.8, 19.5, 20.4, 21.2, 22.6, 24.2, 25.1, 26.0, 26.5, 27.1, 28.4, 29.3, 30.6, 31.2, 33.4, and/or 36.0 degrees 2Θ using Cu Kα radiation, wherein the 2Θ values are ±0.2 degrees 2Θ.

In another embodiment, HBr Form I is characterized as having a powder x-ray diffraction pattern with peaks at 18.8, 21.2, 22.6, 25.0, 26.5 degrees 2Θ using Cu Kα radiation, wherein the 2Θ values are ±0.2 degrees 2Θ.

In another embodiment, HBr Form I is characterized as having a powder x-ray diffraction pattern with peaks at 18.8, 25.0, and 26.5 degrees 2Θ using Cu Kα radiation, wherein the 2Θ values are ±0.2 degrees 2Θ.

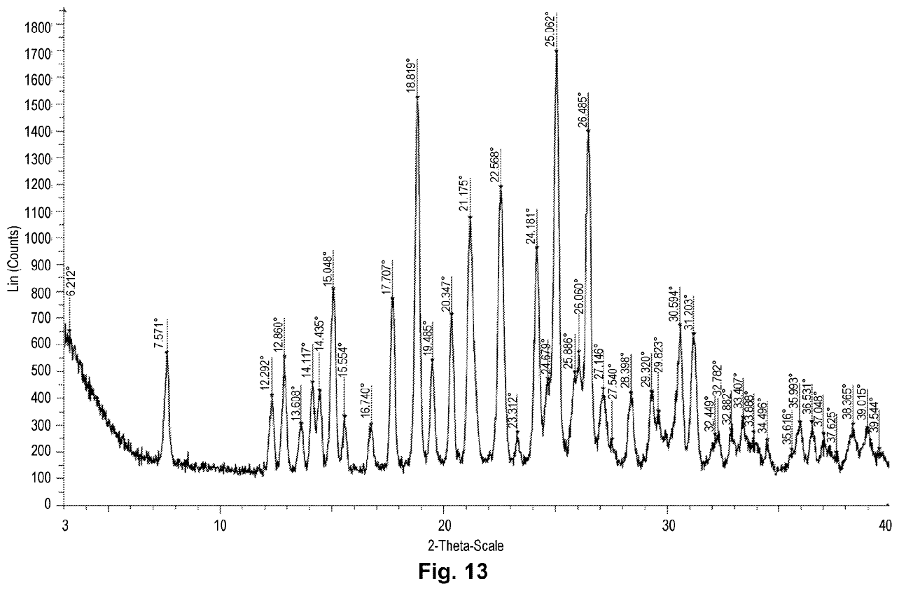

In another embodiment, HBr Form I is characterized as having a XRPD diffractogram that is essentially the same as the one depicted in FIG. 13 using Cu Kα radiation.

In another embodiment, HBr Form I is characterized as having thermal events with an onset temperature of 66.4° C. and a peak temperature of 122.8° C.; and an onset temperature of 177.7° C. and a peak temperature of 189.0° C. based on differential scanning calorimetry (DSC).

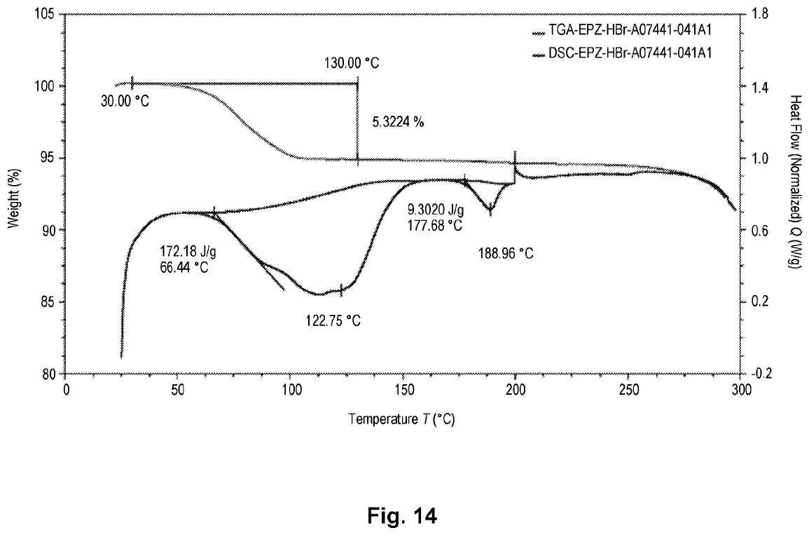

In another embodiment, HBr Form I is characterized as having a DSC thermogram that is essentially the same as the one depicted in FIG. 14.

The crystalline forms of Compound 1, crystalline forms of Compound 1 HCl, and crystalline forms of Compound 1 HBr described in this section are collectively referred to as “Crystalline Forms of the Disclosure” (each individually referred to as a “Crystalline Form of the Disclosure”).

In another embodiment, a Crystalline Form of the Disclosure is characterized as comprising about 10% to about 20%, e.g., about 10%, about 11%, about 12%, about 13%, about 13%, about 14%, about 15%, about 16%, about 17%, about 18%, about 19%, or about 20%, by weight, of another physical form, e.g., crystalline or amorphous forms, of Compound 1, Compound 1 HCl, or Compound 1 HBr.

In another embodiment, a Crystalline Form of the Disclosure is characterized as comprising about 1% to about 10%, e.g., about 10%, about 9%, about 8%, about 7%, about 6%, about 5%, about 4%, about 3%, about 2%, or about 1%, by weight, of another physical form of Compound 1, Compound 1 HCl, or Compound 1 HBr.

In another embodiment, a Crystalline Form of the Disclosure is characterized as comprising about 0.1% to about 1%, e.g., about 1%, about 0.9%, about 0.8%, about 0.7%, about 0.6%, about 0.5%, about 0.4%, about 0.3%, about 0.2%, or about 0.1%, by weight, another physical form of Compound 1, Compound 1 HCl, or Compound 1 HBr.

In another embodiment, a Crystalline Form of the Disclosure is characterized as comprising no XRPD-detectable amount of any other physical forms of Compound 1, Compound 1 HCl, or Compound 1 HBr.

In another embodiment, a Crystalline Form of the Disclosure has an average particle size distribution of about 0.1 μm to about 500 μm.

In another embodiment, a Crystalline Form of the Disclosure has an average particle size distribution of about 1 μm to about 100 μm.

In another embodiment, a Crystalline Form of the Disclosure has an average particle size distribution of about 5 μm to about 25 μm.

In another embodiment, a Crystalline Form of the Disclosure has an average particle size distribution of about 100 μm, about 90 μm, about 80 μm, about 70 μm, about 60 μm, about 50 μm, about 40 μm, about 35 μm, about 30 μm, about 25 μm, about 20 μm, about 15 μm, about 10 μm, about 5 μm, or about 1 μm.

### II. Compositions of the Disclosure

In another embodiment, the present disclosure provides a pharmaceutical composition comprising a Crystalline Form of the Disclosure and one or more pharmaceutically acceptable excipients.

In another embodiment, the one or more pharmaceutically acceptable excipients comprise a ductile diluent, e.g., microcrystalline cellulose, partially pregelatinized maize starch; a brittle diluent, e.g., anhydrous lactose, mannitol, anhydrous dicalcium phosphate; a disintegrant, e.g., croscarmellose sodium, sodium starch glycolate, crospovidone; a binder, e.g., hydroxypropyl cellulose, a glidant, e.g., colloidal silicon dioxide; or a lubricant, e.g., magnesium stearate, stearic acid; or a combination thereof.

In another embodiment, the pharmaceutical composition comprises (a) about 20% w/w to about 30% w/w of the crystalline form; (b) about 60% w/w to about 80% w/w of one or more ductile or brittle diluents; (c) about 1% w/w to about 5% w/w of one or more disintegrants; (d) about 0.5% w/w to about 3% w/w of one or more lubricants.

In another embodiment, the one or more pharmaceutically acceptable excipients comprise microcrystalline cellulose, partially pregelatinized maize starch, anhydrous lactose, mannitol, anhydrous dicalcium phosphate, croscarmellose sodium, sodium starch glycolate, crospovidone, hydroxypropyl cellulose, colloidal silicon dioxide, magnesium stearate, or stearic acid, or a combination thereof.

In another embodiment, the pharmaceutical composition comprises (a) about 25% w/w of the crystalline form; (b) about 35% w/w of microcrystalline cellulose; (c) about 35% w/w of anhydrous lactose; (d) about 3% w/w of croscarmellose sodium; (e) about 1.5% w/w of magnesium stearate.

In another embodiment, the pharmaceutical composition is a dry granule.

In another embodiment, the pharmaceutical composition is formulated as a tablet.

In another embodiment, the pharmaceutical composition is formulated as a film coated tablet.

In another embodiment, the film coating comprises hydroxypropylmethylcellulose (HPMC) 2910/hypromellose, titanium dioxide and macrogol/PEG.

The pharmaceutical compositions and formulations described in this section are collectively referred to as “Compositions of the Disclosure” (each individually referred to as a “Composition of the Disclosure”).

### III. Methods of Making a Composition of the Disclosure

In another embodiment, the present disclosure provides methods of making a Composition of the Disclosure, the method comprising blending a Crystalline Form of the Disclosure with the one or more pharmaceutically acceptable excipients.

In another embodiment, the method of making a Composition of the Disclosure comprises screening the one or more pharmaceutically acceptable excipients to remove any potential agglomeration, e.g., through a 40 mesh or 50 mesh screen depending on the excipient.

In another embodiment, the method of making a Composition of the Disclosure comprises blending the one or more excipients, e.g., at a blending speed of 20 rpm for 10 to 30 minutes, to give a uniform blend.

In another embodiment, the method of making a Composition of the Disclosure comprises lubricating the blend, e.g., with magnesium stearate or stearic acid, e.g., at a blending spend of 20 rpm for 5 to 10 minutes, to give a lubricated blend.

In another embodiment, the method of making a Composition of the Disclosure comprises passing the lubricated blend through a roller compactor to produce dry grannules. In another embodiment, the dry grannules are lubricated, e.g., with magnesium stearate or stearic acid, e.g., at a blending spend of 20 rpm for 5 to 10 minutes, to give lubricated grannules.

In another embodiment, the present disclosure provides methods of making a Composition of the Disclosure, the method comprising compressing the lubricated grannules to give a tablet.

In another embodiment, the tablet is coated with a film coating composition. Suitable film coatings are described in Table 1 and in WO 2013/045961.

TABLE 1

| | | Feature of |
|---|---|---|
| Chemical Name | Brand Name | Coating |
| Ethylcellulose | Ethocel | water-insoluble |
| | Aquacoat ® ECD | |
| | Surelease | |
| Polyvinylacetate | Kollicoat ® SR | water-insoluble |
| Methacrylate copolymers | Eudragit ® NE, RL, RS | water-insoluble |
| | Kollicoat ®EMM | |
| | Eudragit ® L, S, FS | enteric |
| | Kollicoat ® MAE | |
| | Eudragit E | stomach-soluble |
| Cellulose half esters | Aquacoat ® CPD | enteric |
| Hydroxypropylmethyl- | Pharmacoat 606 | water-soluble |
| cellulose (HPMC) | Sepifilm 752 | |
| | Opadry 1 | |
| Polyvinylalcohol (PVA) | Opadry II Clear | water-soluble |
| | Opadry II White | |

In another embodiment, the film coating composition comprises HPMC 2910/hypromellose, titanium dioxide, and macrogol/PEG.

### IV. Methods of Treating a Disease, Disorder, or Condition

In another embodiment, the present disclosure provides a method of treating a disease, disorder or condition in a subject in need thereof, the method comprising administering a therapeutically effective amount of a Crystalline Form of the Disclosure, or a therapeutically amount of Composition of the Disclosure to the subject.

In another embodiment, the disease, disorder or condition is cancer.

In another embodiment, the cancer is any one or more of the cancers of Table 2.

TABLE 2

| | |
|---|---|
| adrenal cancer | lymphoepithelioma |
| acinic cell carcinoma | lymphoma |
| acoustic neuroma | acute lymphocytic leukemia |
| acral lentigious melanoma | acute myelogeous leukemia |
| acrospiroma | chronic lymphocytic leukemia |
| acute eosinophilic leukemia | liver cancer |
| acute erythroid leukemia | small cell lung cancer |
| acute lymphoblastic leukemia | non-small cell lung cancer |
| acute megakaryoblastic leukemia | MALT lymphoma |
| acute monocytic leukemia | malignant fibrous histiocytoma |
| acute promyelocytic leukemia | malignant peripheral nerve sheath |
| | tumor |
| adenocarcinoma | malignant triton tumor |
| adenoid cystic carcinoma | mantle cell lymphoma |
| adenoma | marginal zone B-cell lymphoma |
| adenomatoid odontogenic tumor | mast cell leukemia |
| adenosquamous carcinoma | mediastinal germ cell tumor |
| adipose tissue neoplasm | medullary carcinoma of the breast |
| adrenocortical carcinoma | medullary thyroid cancer |
| adult T-cell leukemia/lymphoma | medulloblastoma |
| aggressive NK-cell leukemia | melanoma |
| AIDS-related lymphoma | meningioma |
| alveolar rhabdomyosarcoma | merkel cell cancer |
| alveolar soft part sarcoma | mesothelioma |
| ameloblastic fibroma | metastatic urothelial carcinoma |
| anaplastic large cell lymphoma | mixed Mullerian tumor |
| anaplastic thyroid cancer | mucinous tumor |
| angioimmunoblastic T-cell | multiple myeloma |
| lymphoma | |
| angiomyolipoma | muscle tissue neoplasm |
| angiosarcoma | mycosis fungoides |
| astrocytoma | myxoid liposarcoma |
| atypical teratoid rhabdoid tumor | myxoma |
| B-cell chronic lymphocytic leukemia | myxosarcoma |
| B-cell prolymphocytic leukemia | nasopharyngeal carcinoma |
| B-cell lymphoma | neurinoma |
| basal cell carcinoma | neuroblastoma |
| biliary tract cancer | neurofibroma |
| bladder cancer | neuroma |
| blastoma | nodular melanoma |
| bone cancer | ocular cancer |
| Brenner tumor | oligoastrocytoma |
| Brown tumor | oligodendroglioma |
| Burkitt's lymphoma | oncocytoma |
| breast cancer | optic nerve sheath meningioma |
| brain cancer | optic nerve tumor |
| carcinoma | oral cancer |
| carcinoma in situ | osteosarcoma |
| carcinosarcoma | ovarian cancer |
| cartilage tumor | Pancoast tumor |
| cementoma | papillary thyroid cancer |
| myeloid sarcoma | paraganglioma |
| chondroma | pinealoblastoma |
| chordoma | pineocytoma |
| choriocarcinoma | pituicytoma |
| choroid plexus papilloma | pituitary adenoma |
| clear-cell sarcoma of the kidney | pituitary tumor |
| craniopharyngioma | plasmacytoma |
| cutaneous T-cell lymphoma | polyembryoma |
| cervical cancer | precursor T-lymphoblastic |
| | lymphoma |
| colorectal cancer | primary central nervous system |
| | lymphoma |
| Degos disease | primary effusion lymphoma |
| desmoplastic small round cell tumor | preimary peritoneal cancer |
| diffuse large B-cell lymphoma | prostate cancer |
| dysembryoplastic neuroepithelial | pancreatic cancer |
| tumor | |
| dysgerminoma | pharyngeal cancer |
| embryonal carcinoma | pseudomyxoma periotonei |
| endocrine gland neoplasm | renal cell carcinoma |
| endodermal sinus tumor | renal medullary carcinoma |
| enteropathy-associated T-cell | retinoblastoma |
| lymphoma | |
| esophageal cancer | rhabdomyoma |
| fetus in fetu | rhabdomyosarcoma |
| fibroma | Richter's transformation |
| fibrosarcoma | rectal cancer |
| follicular lymphoma | sarcoma |
| follicular thyroid cancer | Schwannomatosis |
| ganglioneuroma | seminoma |
| gastrointestinal cancer | Sertoli cell tumor |
| germ cell tumor | sex cord-gonadal stromal tumor |
| gestational choriocarcinoma | signet ring cell carcinoma |
| giant cell fibroblastoma | skin cancer |
| giant cell tumor of the bone | small blue round cell tumors |
| glial tumor | small cell carcinoma |
| glioblastoma multiforme | soft tissue sarcoma |
| glioma | somatostatinoma |
| gliomatosis cerebri | soot wart |
| glucagonoma | spinal tumor |
| gonadoblastoma | splenic marginal zone lymphoma |
| granulosa cell tumor | squamous cell carcinoma |
| gynandroblastoma | synovial sarcoma |
| gallbladder cancer | Sezary's disease |
| gastric cancer | small intestine cancer |
| hairy cell leukemia | squamous carcinoma |
| hemangioblastoma | stomach cancer |
| head and neck cancer | T-cell lymphoma |
| hemangiopericytoma | testicular cancer |
| hematological malignancy | thecoma |
| hepatoblastoma | thyroid cancer |
| hepatosplenic T-cell lymphoma | transitional cell carcinoma |
| Hodgkin's lymphoma | throat cancer |
| non-Hodgkin's lymphoma | urachal cancer |
| invasive lobular carcinoma | urogenital cancer |
| intestinal cancer | urothelial carcinoma |
| kidney cancer | uveal melanoma |
| laryngeal cancer | uterine cancer |
| lentigo maligna | verrucous carcinoma |
| lethal midline carcinoma | visual pathway glioma |
| leukemia | vulvar cancer |
| leydig cell tumor | vaginal cancer |
| liposarcoma | Waldenstrom's macroglobulinemia |
| lung cancer | Warthin's tumor |
| lymphangioma | Wilms' tumor. |
| lymphangiosarcoma | |

In another embodiment, the cancer is a hematological cancer. Exemplary hematological cancers include, but are not limited to, the cancers listed in Table 3.

TABLE 3

| | |
|---|---|
| acute lymphocytic leukemia (ALL) | acute eosinophilic leukemia |
| acute myeloid leukemia (AML) | acute erythroid leukemia |
| chronic lymphocytic leukemia (CLL) | acute lymphoblastic leukemia |
| small lymphocytic lymphoma (SLL) | acute megakaryoblastic leukemia |
| multiple myeloma (MM) | acute monocytic leukemia |
| Hodgkins lymphoma (HL) | acute promyelocytic leukemia |
| non-Hodgkin's lymphoma (NHL) | acute myelogeous leukemia |
| mantle cell lymphoma (MCL) | B-cell prolymphocytic leukemia |
| marginal zone B-cell lymphoma | B-cell lymphoma |
| splenic marginal zone lymphoma | MALT lymphoma |
| follicular lymphoma (FL) | precursor T-lymphoblastic |
| | lymphoma |
| Waldenstrom's macroglobulinemia | T-cell lymphoma |
| (WM) | |
| diffuse large B-cell lymphoma | mast cell leukemia |
| (DLBCL) | |
| marginal zone lymphoma (MZL) | adult T cell leukemia/lymphoma |
| hairy cell leukemia (HCL) | aggressive NK-cell leukemia |
| Burkitt's lymphoma (BL) | angioimmunoblastic T-cell |
| | lymphoma |
| Richter's transformation | |

In another embodiment, the cancer is diffuse large B-cell lymphoma.

In another embodiment, the cancer is mantle cell lymphoma.

In another embodiment, the cancer is multiple myeloma.

In another embodiment, the multiple myeloma is characterized as having chromosomal translocations involving the immunoglobulin heavy chain locus at 14q32.

In another embodiment, the chromosomal translocation is a t(4;14) translocation, i.e., the multiple myeloma is t(4;14) multiple myeloma.

In another embodiment, the present disclosure provides a method of treating cancer in a subject in need thereof, the method comprising administering a therapeutically effective amount of a Crystalline Form of the Disclosure, or a therapeutically amount of Composition of the Disclosure to the subject in combination with a therapeutically effective amount of an anti-cancer agent to the subject.

In another embodiment, the anti-cancer agent comprises one or more glucocorticoid receptor agonists, one or more immunomodulatory drugs, one or more proteasome inhibitors, one or more Bcl-2 inhibitors, one or more pleiotropic pathway modulators, one or more XPO1 inhibitors, one or more histone deacetylase inhibitors, one or more EZH2 inhibitors, one or more BTK inhibitors, one or more anti-CD20 monoclonal antibodies, one or more alkylating agents, one or more topoisomerase II inhibitors, one or more vinca alkaloids, one or more platinum-based drugs, one or more nucleoside anticancer agents, one or more PI3K inhibitors, one or more CDK4/6 inhibitors, or one or more CARM1 inhibitors, or a combination thereof.

In another embodiment, the anti-cancer agent comprises a glucocorticoid receptor agonist. In another embodiment, the glucocorticoid receptor agonist is dexamethasone.

In another embodiment, the anti-cancer agent comprises an immunomodulatory drug. In another embodiment, the immunomodulatory drug is pomalidomide or lenalidomide.

In another embodiment, the anti-cancer agent comprises a proteasome inhibitor. In another embodiment, the proteasome inhibitor is bortezomib.

In another embodiment, the anti-cancer agent comprises a Bcl-2 inhibitor. In another embodiment, the Bcl-2 inhibitor is venetoclax.

In another embodiment, the anti-cancer agent comprises a pleiotropic pathway modulator. In another embodiment, the pleiotropic pathway modulator is CC-122.

In another embodiment, the anti-cancer agent comprises a XPO1 inhibitor. In another embodiment, the XPO1 inhibitor is selinexor.

In another embodiment, the anti-cancer agent comprises a histone deacetylase inhibitor. In another embodiment, the histone deacetylase inhibitor is panobinostat.

In another embodiment, the anti-cancer agent is an EZH2 inhibitor. In another embodiment, the EZH2 inhibitor is tazemetostat.

In another embodiment, the anti-cancer agent comprises a BTK inhibitor. In another embodiment, the BTK inhibitor is ibrutinib, acalabrutinib, or zanubrutinib.

In another embodiment, the anti-cancer agent comprises an anti-CD20 monoclonal antibody. In another embodiment, the anti CD20 monoclonal antibody is rituximab.

In another embodiment, the anti-cancer agent comprises a PI3K inhibitor. In another embodiment, the PI3K inhibitor is copanlisib.

In another embodiment, the anti-cancer agent comprises a CDK4/6 inhibitor. In another embodiment, the CDK4/6 inhibitor is palbociclib.

In another embodiment, the anti-cancer agent comprises a CARM1 inhibitor. In another embodiment, the CARM1 inhibitor is EZM2302.

In another embodiment, the anti-cancer agent comprises an alkylating agent. In another embodiment, the alkylating agent is mafosfamide.

In another embodiment, the anti-cancer agent comprises a topoisomerase II inhibitor. In another embodiment, the topoisomerase II inhibitor is doxorubicin and etoposide.

In another embodiment, the anti-cancer agent comprises a vinca alkaloid. In another embodiment, the vinca alkaloid is vincristine.

In another embodiment, the anti-cancer agent comprises a platinum-based drug. In another embodiment, the platinum-based drug is carboplatin or oxaliplatin.

In another embodiment, the anti-cancer agent comprises a nucleoside anticancer agent. In another embodiment, the nucleoside anticancer agent is gemcitabine.

### V. Kits

In another embodiment, the present disclosure provides a kit comprising a Crystalline Form of the Disclosure or Composition of the Disclosure packaged in a manner that facilitates its use to practice methods of the present disclosure.

In another embodiment, the kit includes a Crystalline Form of the Disclosure or Composition of the Disclosure packaged in a container, such as a sealed bottle, with a label affixed to the container and an insert included in the kit that describes the use of the Crystalline Form of the Disclosure or Composition of the Disclosure to practice a method of the disclosure for treating a disease, disorder, or condition, e.g., cancer, in a subject. In another embodiment, the Crystalline Form of the Disclosure or Composition of the Disclosure is packaged in a unit dosage form, e.g., as a tablet, e.g., as a film coated tablet.

In another embodiment, the kit further comprises an insert, e.g., instructions for administering the Crystalline Form of the Disclosure or Composition of the Disclosure to a subject having a disease, disorder or condition. In another embodiment, the disease, disorder or condition is cancer.

### VI. Methods of Making a Crystalline Form of the Disclosure

In another embodiment, the present disclosure provides methods of making a Crystalline Form of the Disclosure.

In another embodiment, the present disclosure provides a method of making Free Base Form II.

In another embodiment, the present disclosure provides a method of making Free Base Form II, the method comprising:
(i) heating a mixture of Compound 1, ethanol, and water until Compound 1 is dissolved;
(ii) cooling the solution to about 50° C.;
(iii) optionally seeding the solution with Free Base Form II;
(iv) cooling the solution to about 0° C.; and
(v) isolating the Free Base Form II thus formed.

In another embodiment, the Free Base Form II is isolated by filtration.

In another embodiment, the method of further comprises washing the Free Base Form II with a mixture of water and ethanol.

In another embodiment, the method further comprises drying the Free Base Form II under vacuum.

### VII. Definitions

N-((1R,3S)-3-(4-acetylpiperazin-1-yl)cyclohexyl)-4-fluoro-7-methyl-1H-indole-2-carboxamide or “Compound 1” refers to a compound having the structure:

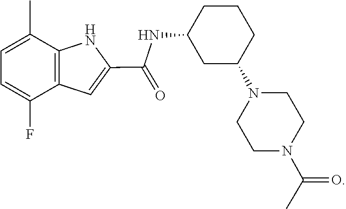 [SMILES: CC(=O)N1CCN([C@H]2CCC[C@@H](NC(=O)c3cc4c(F)ccc(C)c4[nH]3)C2)CC1]

N-((1R,3S)-3-(4-acetylpiperazin-1-yl)cyclohexyl)-4-fluoro-7-methyl-1H-indole-2-carboxamide hydrochloride or “Compound 1 HCl” refers to the hydrochloric acid salt of N-((1R,3S)-3-(4-acetylpiperazin-1-yl)cyclohexyl)-4-fluoro-7-methyl-1H-indole-2-carboxamide.

N-((1R,3S)-3-(4-acetylpiperazin-1-yl)cyclohexyl)-4-fluoro-7-methyl-1H-indole-2-carboxamide hydrobromide or “Compound 1 HBr” refers to the hydrobromic acid salt of N-((1R,3S)-3-(4-acetylpiperazin-1-yl)cyclohexyl)-4-fluoro-7-methyl-1H-indole-2-carboxamide.

As used herein, the term “substantially pure” with reference to a Crystalline Form of the Disclosure means that the crystalline material comprises about 10% or less, e.g., about 1% to about 10%, e.g., about 9%, about 8%, about 7%, about 6%, about 5%, about 4%, about 3%, about 2%, or about 1%, by weight of any other crystalline or amorphous form(s) of Compound 1, Compound 1 HCl, or Compound 1 HBr. In another embodiment, the Crystalline Form of the Disclosure is substantially pure Free Base Form II.

As used herein, the term “pure” with reference to a Crystalline Form of the Disclosure means that the crystalline material comprises about 1% or less, e.g., about 0.1% to about 1%, e.g., about 1%, about 0.9%, about 0.8%, about 0.7%, about 0.6%, about 0.5%, about 0.4%, about 0.3%, about 0.2%, or about 0.1%, or less, by weight of any other crystalline or amorphous form(s) of Compound 1, Compound 1 HCl, or Compound 1 HBr. In one embodiment, the Crystalline Form of the Disclosure contains no XRPD-detectable amount of any other crystalline or amorphous form(s) of Compound 1, Compound 1 HCl, or Compound 1 HBr.

As used herein, the term “amorphous” refers to a solid form of Compound 1, Compound 1 HCl, or Compound 1 HBr that lacks the long-range order characteristic of a crystal, i.e., the solid is non-crystalline.

As used herein, the term “essentially the same” with reference to XRPD peak positions and/or relative intensities means that peak position and/or intensity variabilities are taken into account when comparing XRPD diffractograms. Likewise, term “essentially the same” with reference to Raman or IR peak positions means that peak position variabilities are taken into account when comparing Raman or IR spectra. For example, XRPD peak positions can show, e.g., inter-apparatus variability, e.g., as much as 0.2° 2Θ, i.e., ±0.2 degrees 2Θ; Raman and IR peak positions can show, e.g., inter-apparatus variability, e.g., as much 4 cm⁻¹, i.e., ±4 cm⁻¹. Relative peak intensities, for example, in a XRPD diffractogram, can also show inter-apparatus variability due to degree of crystallinity, orientation, prepared sample surface, and other factors known to those skilled in the art, and should be taken as qualitative measures only.

As used herein, the term “micronization” refers to a process or method by which the size of a population of particles is reduced, typically to the micron scale.

As used herein, the term “micron” or “μm” refer to “micrometer,” which is 1×10⁻⁶ meter.

As used herein, the term “therapeutically effective amount,” refers to the amount of Compound 1 sufficient to treat one or more symptoms of a disease, condition, injury, or disorder, or prevent advancement of disease, condition, injury, or disorder, or cause regression of the disease, condition, injury, or disorder.

In one embodiment, a Crystalline Form of the Disclosure or Composition of the Disclosure is administered to the subject in an amount that is effective to achieve its intended therapeutic purpose. While individual needs may vary, a determination of optimal ranges of effective amounts of each compound is within the skill of the art. Typically, a Crystalline Form of the Disclosure is administered to a mammal, e.g., a human, orally at a dose of from about 0.0025 to about 1500 mg per kg body weight of the mammal, or an equivalent amount of a pharmaceutically acceptable salt or solvate thereof, per day to treat the particular disorder. A useful oral dose of a Crystalline Form of the Disclosure administered to a mammal is from about 0.1 mg to about 10 mg per kg body weight of the mammal, or an equivalent amount of the pharmaceutically acceptable salt or solvate thereof.

In one embodiment, a Crystalline Form of the Disclosure or Composition of the Disclosure is administered to the subject in a total daily dose of from about 50 mg to about 2000 mg. In another embodiment, a Crystalline Form of the Disclosure or Composition of the Disclosure is administered to the subject in a total daily dose of from about 100 mg to about 1000 mg. In another embodiment, a Crystalline Form of the Disclosure or Composition of the Disclosure is administered to the subject in a total daily dose of about 100 mg, about 200 mg, about 300 mg, about 400 mg, about 500 mg, about 600 mg, about 700 mg, about 800 mg, about 900 mg, or about 1000 mg.

A unit oral dose may comprise from about 1 mg to about 1000 mg of a Crystalline Form of the Disclosure, e.g., about 1 mg to about 500 mg, about 10 mg to about 250 mg, about 25 mg to about 200 mg of the crystalline form. The unit dose can be administered one or more times daily, e.g., as one or more tablets or capsules, each containing from about 10 mg to about 250 mg of the compound, or an equivalent amount of a pharmaceutically acceptable salt or solvate thereof. In one embodiment, the unit oral dose, e.g., tablet, comprises about 25 mg of a Crystalline Form of the Disclosure. In another embodiment, the unit oral dose, e.g., tablet, comprises about 100 mg of a Crystalline Form of the Disclosure. In another embodiment, the unit oral dose, e.g., tablet, comprises about 200 mg of a Crystalline Form of the Disclosure. In another embodiment, a unit oral dose of about 10 mg to about 250 mg of a Crystalline Form of the Disclosure is administered to a subject once a day. In another embodiment, a unit oral dose of about 10 mg to about 250 mg of a Crystalline Form of the Disclosure is administered to a subject twice a day. In another embodiment, a unit oral dose of about 10 mg to about 250 mg of a Crystalline Form of the Disclosure is administered to a subject three times a day.

As used herein, the term “chemically stable” and the like with reference to a Crystalline Form of the Disclosure means that the crystalline solid shows less than 0.5% chemical degradation, e.g., less than 0.4%, less than 0.3%, less than 0.2%, less than 0.1%, or less than 0.05% chemical degradation, after storage at temperature of about 25° C. and a relative humidity of about 60% for at least 3 months In determining the amount of degradation, the appearance of one or more chemical impurities can be measured and/or the disappearance of Compound 1 can be measured using methods, e.g., HPLC, known in the art.

The terms “a” and “an” refer to one or more than one.

The term “about” as used herein, includes the recited number ±10%. Thus, “about 10” means 9 to 11.

As used herein, the term “average particle size distribution” or “D50” is the diameter where 50 mass-% of the particles have a larger equivalent diameter, and the other 50 mass-% have a smaller equivalent diameter as determined by laser diffraction, e.g., in Malvern Master Sizer Microplus equipment or its equivalent.

As used herein, the term “excipient” refers to any ingredient in or added to give a pharmaceutical formulation suitable for administration to a subject, e.g., a tablet for oral administration, other than the Crystalline Form of the Disclosure. An excipient is typically an inert substance, e.g., microcrystalline cellulose, added to a composition to facilitate processing, handling, dissolution, administration, etc. of the Crystalline Form of the Disclosure. Useful excipients include, but are not limited to, adjuvants, antiadherents, binders, carriers, disintegrants, fillers, flavors, colors, diluents, lubricants, glidants, preservatives, sorbents, solvents, surfactants, and sweeteners.

Conventional pharmaceutical excipients are well known to those skilled in the art. A wide variety of pharmaceutical excipients can be used in admixture with a Crystalline Form of the Disclosure, including, e.g., microcrystalline cellulose, anhydrous lactose, croscarmellose sodium, and others listed in the Handbook of Pharmaceutical Excipients, Pharmaceutical Press 4th Ed. (2003), and Remington: The Science and Practice of Pharmacy, Lippincott Williams & Wilkins, 21st ed. (2005). In one embodiment, the composition comprises Free Base Form II formulated as a tablet.

As used herein, the term “subject” refers to an animal, e.g., human or veterinary animal, e.g., cow, sheep, pig, horse, dog, or cat. In one embodiment, the subject is a human.

As used herein, the term “container” means any receptacle and closure therefore suitable for storing, shipping, dispensing, and/or handling a pharmaceutical product or excipient.

The term “insert” means information accompanying a pharmaceutical product that provides a description of how to administer the product, along with the safety and efficacy data required to allow the physician, pharmacist, and patient to make an informed decision regarding use of the product. The package insert generally is regarded as the “label” for a pharmaceutical product.

### VIII. Particular Embodiments

The disclosure provides the following particular embodiments.

Embodiment 1. A crystalline polymorph of:
(a) N-((1R,3S)-3-(4-acetylpiperazin-1-yl)cyclohexyl)-4-fluoro-7-methyl-1H-indole-2-carboxamide, having Free Base Form II, Free Base Form III, or Free Base Form IV, or a mixture thereof; or
(b) N-((1R,3S)-3-(4-acetylpiperazin-1-yl)cyclohexyl)-4-fluoro-7-methyl-1H-indole-2-carboxamide hydrochloride, having HCl Form I or HCl Form II, or a mixture thereof; or
(c) N-((1R,3S)-3-(4-acetylpiperazin-1-yl)cyclohexyl)-4-fluoro-7-methyl-1H-indole-2-carboxamide hydrobromide, having HBr Form I,
wherein:
(i) Free Base Form II is characterized as having a powder x-ray diffraction pattern with peaks at 7.0, 14.0, 18.0, and 20.2 degrees 2Θ using Cu Kα radiation, wherein the 20 values are ±0.2 degrees 2Θ;
(ii) Free Base Form III is characterized as having a powder x-ray diffraction pattern with peaks 7.6, 14.8, 18.0, and 19.8 degrees 2Θ using Cu Kα radiation, wherein the 2Θ values are ±0.2 degrees 2Θ;
(iii) Free Base Form IV is characterized as having a powder x-ray diffraction pattern with peaks at 14.8, 18.1, 19.1, 19.9, and 20.5 degrees 2Θ using Cu Kα radiation, wherein the 2Θ values are ±0.2 degrees 2Θ;
(iv) HCl Form I is characterized as having a powder x-ray diffraction pattern with peaks at 14.6 and 25.0 degrees 2Θ using Cu Kα radiation, wherein the 2Θ values are ±0.2 degrees 2Θ;
(v) HCl Form II is characterized as having a powder x-ray diffraction pattern with peaks at 15.2, 16.0, 17.7, and 22.6 degrees 2Θ using Cu Kα radiation, wherein the 20 values are ±0.2 degrees 2Θ; and
(vi) HBr Form I is characterized as having a powder x-ray diffraction pattern with peaks at 18.8, 25.1, and 26.5 degrees 2Θ using Cu Kα radiation, wherein the 2Θ values are ±0.2 degrees 2Θ.

Embodiment 2. The crystalline polymorph of Embodiment 1 which is N-((1R,3S)-3-(4-acetylpiperazin-1-yl)cyclohexyl)-4-fluoro-7-methyl-1H-indole-2-carboxamide having Free Base Form II.

Embodiment 3. The crystalline polymorph of Embodiment 1 which is N-((1R,3S)-3-(4-acetylpiperazin-1-yl)cyclohexyl)-4-fluoro-7-methyl-1H-indole-2-carboxamide having Free Base Form III.

Embodiment 4. The crystalline polymorph of Embodiment 1 which is N-((1R,3S)-3-(4-acetylpiperazin-1-yl)cyclohexyl)-4-fluoro-7-methyl-1H-indole-2-carboxamide having Free Base Form IV.

Embodiment 5. The crystalline polymorph of any one of Embodiments 2-4 comprising about 5% or less of any other physical form of N-((1R,3S)-3-(4-acetylpiperazin-1-yl)cyclohexyl)-4-fluoro-7-methyl-1H-indole-2-carboxamide.

Embodiment 6. The crystalline polymorph of Embodiment 1 which is N-((1R,3S)-3-(4-acetylpiperazin-1-yl)cyclohexyl)-4-fluoro-7-methyl-1H-indole-2-carboxamide hydrochloride having HCl Form I.

Embodiment 7. The crystalline polymorph of Embodiment 1 which is N-((1R,3S)-3-(4-acetylpiperazin-1-yl)cyclohexyl)-4-fluoro-7-methyl-1H-indole-2-carboxamide hydrochloride having HCl Form II.

Embodiment 8. The crystalline polymorph of Embodiments 6 or 7 comprising about 5% or less of any other physical forms of N-((1R,3S)-3-(4-acetylpiperazin-1-yl)cyclohexyl)-4-fluoro-7-methyl-1H-indole-2-carboxamide hydrochloride.

Embodiment 9. The crystalline polymorph of Embodiment 1 which is N-((1R,3S)-3-(4-acetylpiperazin-1-yl)cyclohexyl)-4-fluoro-7-methyl-1H-indole-2-carboxamide hydrochloride having HBr Form I.

Embodiment 10. The crystalline polymorph of Embodiment 9 comprising about 5% or less of any other physical forms of N-((1R,3S)-3-(4-acetylpiperazin-1-yl)cyclohexyl)-4-fluoro-7-methyl-1H-indole-2-carboxamide hydrobromide.

Embodiment 11. A pharmaceutical composition comprising the crystalline polymorph of any one of Embodiments 1-10 and one or more pharmaceutically acceptable excipients.

Embodiment 12. The pharmaceutical composition of Embodiment 11, wherein the one or more pharmaceutically acceptable excipients comprise a ductile diluent, a brittle diluent, a disintegrant, a binder, a glidant, or a lubricant, or a combination thereof.

Embodiment 13. The pharmaceutical composition of Embodiment 11, wherein the one or more pharmaceutically acceptable excipients comprise microcrystalline cellulose, partially pregelatinized maize starch, anhydrous lactose, mannitol, dicalcium phosphate anhydrous, croscarmellose sodium, sodium starch glycolate, crospovidone, hydroxypropyl cellulose, colloidal silicon dioxide, magnesium stearate, or stearic acid, or a combination thereof.

Embodiment 14. The pharmaceutical composition of Embodiment 13, comprising:
(a) about 25% w/w of the crystalline polymorph;
(b) about 35% w/w of microcrystalline cellulose;
(c) about 35% w/w of anhydrous lactose;
(d) about 3% w/w of croscarmellose sodium; and
(e) about 1.5% w/w of magnesium stearate.

Embodiment 15. The pharmaceutical composition of Embodiment 14 further comprising a film coating.

Embodiment 16. The pharmaceutical composition of Embodiment 15, wherein the film coating composition comprises HPMC 2910/hypromellose, titanium dioxide, and macrogol/PEG.

Embodiment 17. The pharmaceutical composition of any one of Embodiments 11-16 formulated as a film coated tablet.

Embodiment 18. A method of making the pharmaceutical composition of any one of Embodiments 11-17, the method comprising blending the crystalline polymorph with the one or more pharmaceutically acceptable excipients.

Embodiment 19. A method of treating cancer in a subject in need thereof, the method comprising administering a therapeutically effective amount of the crystalline polymorph of any one of Embodiments 1-10, or a therapeutically effective amount of the pharmaceutical composition of any one of Embodiments 11-17 to the subject.

Embodiment 20. The crystalline polymorph of any one of Embodiments 1-10, or the pharmaceutical composition of any one of Embodiments 11-17 for use in treating cancer in a subject.

Embodiment 21. Use of the crystalline polymorph of any one of Embodiments 1-10, or the pharmaceutical composition of any one of Embodiments 11-17 in the manufacture of a medicament for treating cancer in a subject

Embodiment 22. The method of Embodiment 19, crystalline polymorph or pharmaceutical composition of claim 20, or use of claim 21, wherein the cancer is any one or more of the cancers of Table 2.

Embodiment 23. The method of Embodiment 21, crystalline polymorph or pharmaceutical composition of claim 20, or use of claim 21, wherein the cancer is a hematological cancer.

Embodiment 24. The method, crystalline polymorph, pharmaceutical composition, or use of Embodiment 23, wherein the hematological cancer is any one or more of the cancers of Table 3.

Embodiment 25. The method, crystalline polymorph, pharmaceutical composition, or use of Embodiment 24, wherein the hematological cancer is diffuse large B-cell lymphoma.

Embodiment 26. The method, crystalline polymorph, pharmaceutical composition, or use of Embodiment 24, wherein the hematological cancer is mantle cell lymphoma.

Embodiment 27. The method, crystalline polymorph, pharmaceutical composition, or use of Embodiment 24, wherein the hematological cancer is multiple myeloma.

Embodiment 28. The method, crystalline polymorph, pharmaceutical composition, or use of Embodiment 27, wherein the hematological cancer is t(4;14) multiple myeloma.

Embodiment 29. The method, crystalline polymorph, pharmaceutical composition, or use of any one of Embodiments 19-28 further comprising administering a therapeutically effective amount of an anti-cancer agent to the subject.

Embodiment 30. The method, crystalline polymorph, pharmaceutical composition, or use of Embodiment 29, where the anti-cancer agent comprises one or more glucocorticoid receptor agonists, one or more immunomodulatory drugs, one or more proteasome inhibitors, one or more Bcl-2 inhibitors, one or more pleiotropic pathway modulators, one or more XPO1 inhibitors, one or more histone deacetylase inhibitors, one or more EZH2 inhibitors, one or more BTK inhibitors, one or more anti-CD20 monoclonal antibodies, one or more alkylating agents, one or more topoisomerase II inhibitors, one or more vinca alkaloids, one or more platinum-based drugs, one or more nucleoside anticancer agents, one or more PI3K inhibitors, one or more CDK4/6 inhibitors, or one or more CARM1 inhibitors, or a combination thereof.

Embodiment 31. The method, crystalline polymorph, pharmaceutical composition, or use Embodiments 29 or 30, wherein the anti-cancer agent comprises a glucocorticoid receptor agonist.

Embodiment 32. The method, crystalline polymorph, pharmaceutical composition, or use of Embodiment 31, wherein the glucocorticoid receptor agonist is dexamethasone.

Embodiment 33. The method, crystalline polymorph, pharmaceutical composition, or use of any one of Embodiments 29-32, wherein the anti-cancer agent comprises an immunomodulatory drug.

Embodiment 34. The method, crystalline polymorph, pharmaceutical composition, or use of Embodiment 33, wherein the immunomodulatory drug is pomalidomide or lenalidomide.

Embodiment 35. The method, crystalline polymorph, pharmaceutical composition, or use of any one of Embodiments 29-34, wherein the anti-cancer Agent comprises a proteasome inhibitor.

Embodiment 36. The method, crystalline polymorph, pharmaceutical composition, or use of Embodiment 35, wherein the proteasome inhibitor is bortezomib.

Embodiment 37. The method, crystalline polymorph, pharmaceutical composition, or use of any one of Embodiments 29-36, wherein the anti-cancer agent comprises a Bcl-2 inhibitor.

Embodiment 38. The method of Embodiment 37, wherein the Bcl-2 inhibitor is venetoclax.

Embodiment 39. The method of any one of Embodiments 29-38, wherein the anti-cancer agent comprises a pleiotropic pathway modulator.

Embodiment 40. The method of Embodiment 39, wherein the pleiotropic pathway modulator is CC-122.

Embodiment 41. The method of any one of Embodiments 29-40, wherein the anti-cancer agent comprises a XPO1 inhibitor.

Embodiment 42. The method of Embodiment 41, wherein the XPO1 inhibitor is selinexor.

Embodiment 43. The method of any one of Embodiments 29-42, wherein the anti-cancer agent comprises a histone deacetylase inhibitor.

Embodiment 44 The method of Embodiment 43, wherein the histone deacetylase inhibitor is panobinostat.

Embodiment 45. The method of any one of Embodiments 29-44, wherein the anti-cancer agent is an EZH2 inhibitor.

Embodiment 46. The method of Embodiment 45, wherein the EZH2 inhibitor is tazemetostat.

Embodiment 47. The method of any one of Embodiments 29-46, wherein the anti-cancer agent comprises a BTK inhibitor.

Embodiment 48. The method of Embodiment 47, wherein the BTK inhibitor is ibrutinib, acalabrutinib, or zanubrutinib.

Embodiment 49. The method of any one of Embodiments 29-48, wherein the anti-cancer agent comprises an anti-CD20 monoclonal antibody.

Embodiment 50. The method of Embodiment 49, wherein the anti CD20 monoclonal antibody is rituximab.

Embodiment 51. The method of any one of Embodiments 29-50, wherein the anti-cancer agent comprises a PI3K inhibitor.

Embodiment 52. The method of Embodiment 51, wherein the PI3K inhibitor is copanlisib.

Embodiment 53. The method of any one of Embodiments 29-52, wherein the anti-cancer agent comprises a CDK4/6 inhibitor.

Embodiment 54 The method of Embodiment 53, wherein the CDK4/6 inhibitor is palbociclib.

Embodiment 55. The method of any one of Embodiments 29-54, wherein the anti-cancer agent comprises a CARM1 inhibitor.

Embodiment 56. The method of Embodiment 55, wherein the CARM1 inhibitor is EZM2302.

Embodiment 57. The method of any one of Embodiments 29-56, wherein the anti-cancer agent comprises an alkylating agent.

Embodiment 58. The method of Embodiment 57, wherein the alkylating agent is mafosfamide.

Embodiment 59. The method of any one of Embodiments 29-58, wherein the anti-cancer agent comprises a topoisomerase II inhibitor.

Embodiment 60. The method of Embodiment 58, wherein the topoisomerase II inhibitor is doxorubicin and etoposide.

Embodiment 61. The method of any one of Embodiments 29-60, wherein the anti-cancer agent comprises a vinca alkaloid.

Embodiment 62. The method of Embodiment 61, wherein the vinca alkaloid is vincristine.

Embodiment 63. The method of any one of Embodiments 29-62, wherein the anti-cancer agent comprises a platinum-based drug.

Embodiment 64. The method of Embodiment 63, wherein the platinum-based drug is carboplatin or oxaliplatin.

Embodiment 65. The method of any one of Embodiments 29-64, wherein the anti-cancer agent comprises a nucleoside anticancer agent.

Embodiment 66. The method of Embodiment 65, wherein the nucleoside anticancer agent is gemcitabine.

Embodiment 67. A kit comprising the crystalline polymorph of any one of Embodiments 1-10, or the pharmaceutical composition of any one of Embodiments 11-17, and instructions for administering the pharmaceutical composition to a subject in need thereof.

Embodiment 68. The kit of Embodiment 67 further comprising an anti-cancer agent.

Embodiment 69. A kit for carrying out the method of any one of Embodiments 19 or 22-28, the kit comprising (a) the crystalline polymorph or the pharmaceutical composition; and (b) instructions for administering the crystalline polymorph or the pharmaceutical composition the subject.

Embodiment 70. A kit for carrying out the method of any one of Embodiments 29-66, the kit comprising (a) the crystalline polymorph or the pharmaceutical composition; (b) instructions for administering the crystalline polymorph or the pharmaceutical composition to the subject; (c) the anti-cancer agent; and (d) instructions for administering the anti-cancer agent to the subject.

Embodiment 71. A method of making the Free Base Form II of Embodiment 1, the method comprising:
(i) heating N-((1R,3S)-3-(4-acetylpiperazin-1-yl)cyclohexyl)-4-fluoro-7-methyl-1H-indole-2-carboxamide in ethanol and water to give a solution;
(ii) cooling the solution to about 0° C.;
(iii) optionally seeding the solution; and
(iv) isolating the Free Base Form II.

### EXAMPLES

### Instrumentation

### Powder X-Ray Diffraction (PXRD or XRPD)

XRPD patterns were identified with an X-ray diffractometer, e.g., a Bruker D8 advance or D2 Phaser equipped with LynxEye detector. Samples were scanned from 3 to 40° 2Θ, at a step size 0.02° 20. The tube voltage and current were 40 KV and 40 mA, respectively.

### Differential Scanning Calorimetry (DSC)

DSC was performed using a Discovery DSC 250 (TA Instruments, US). The sample was placed into an aluminum pin-hole hermetic pan and the weight was accurately recorded. The sample was heated at a rate of 10° C./min from 25° C. to the final temperature.

### Fourier Transform Infrared Spectroscopy (FT-IR)

FTIR was obtained with a Shimadzu IR Tracer 100. Samples were prepared via a KBr pellet method, and the IR spectrum was recorded between 4000-400 cm⁻¹.

### Thermogravimetric Analysis (TGA)

TGA was carried out on a Discovery TGA 55 (TA Instruments, US). The sample was placed into an open tared aluminum pan, automatically weighed, and inserted into the TGA furnace. The sample was heated at a rate of 10° C./min from ambient temperature to the final temperature.

### Example 1

### Synthesis and Characterization of Polymorph of the Disclosure

### Free Base Form I

A racemic mixture of free base of Compound 1 was separated via chiral Supercritical fluid chromatography (SFC), fractions containing Compound 1 were pooled and concentrated under reduced pressure. The material was then dried under vacuum (˜15 Torr) to afford Free Base Form I.

### Free Base Form II

The synthesis of the TFA salt of Compound 1 is described in Example 1 of WO 2020/037079. The free base of Compound 1 is heated in a solution of EtOH and water to dissolve the compound. The solution is slowly cooled to 50±5° C., and seeds are added. The mixture is cooled further to approximately 2.5±2.5° C. The crystalline product was filtered and washed with a mixture of purified water and ethanol, then dried under vacuum to afford Free Base Form II. The XRPD peak list (±0.2 degrees 2Θ) of Free Base Form II is provided in Table A.

TABLE A

| Peak (2Θ °) | Intensity % | Intensity Count | d-spacing Å |
|---|---|---|---|
| 7.0 | 73.4 | 37882 | 12.66 |
| 9.8 | 14.3 | 7383 | 9.05 |
| 10.0 | 7.1 | 3673 | 8.82 |
| 11.0 | 3.9 | 2003 | 8.07 |
| 11.2 | 8.5 | 4404 | 7.91 |
| 14.0 | 61 | 31472 | 6.31 |
| 14.3 | 1.9 | 1004 | 6.18 |
| 15.1 | 2.4 | 1239 | 5.85 |
| 15.5 | 11.8 | 6094 | 5.70 |
| 15.9 | 1.2 | 595 | 5.58 |
| 16.2 | 1 | 491 | 5.46 |
| 16.8 | 1.6 | 823 | 5.29 |
| 17.5 | 4.5 | 2337 | 5.08 |
| 18.0 | 100 | 51606 | 4.92 |
| 19.4 | 2.6 | 1325 | 4.57 |
| 19.6 | 9.2 | 4768 | 4.52 |
| 20.2 | 32.3 | 16661 | 4.40 |
| 20.8 | 11.9 | 6122 | 4.28 |
| 21.1 | 5.6 | 2887 | 4.20 |
| 22.1 | 15.9 | 8202 | 4.02 |
| 22.4 | 12.1 | 6270 | 3.96 |
| 23.8 | 4.4 | 2282 | 3.73 |
| 24.4 | 1.9 | 1005 | 3.65 |
| 24.8 | 1.6 | 848 | 3.59 |
| 25.1 | 4.6 | 2362 | 3.54 |
| 25.7 | 1.6 | 837 | 3.46 |
| 25.9 | 3.7 | 1888 | 3.43 |
| 26.2 | 1 | 515 | 3.39 |
| 27.2 | 0.9 | 454 | 3.28 |
| 27.9 | 0.7 | 343 | 3.19 |
| 28.3 | 4.2 | 2182 | 3.15 |
| 28.6 | 0.9 | 476 | 3.12 |
| 29.4 | 9.3 | 4797 | 3.04 |
| 30.0 | 1.4 | 731 | 2.98 |
| 30.4 | 0.7 | 370 | 2.94 |
| 31.0 | 0.6 | 306 | 2.88 |
| 31.4 | 1.9 | 993 | 2.84 |
| 32.1 | 1 | 521 | 2.79 |
| 32.7 | 0.6 | 332 | 2.74 |
| 33.1 | 1 | 518 | 2.71 |
| 33.4 | 1.1 | 570 | 2.68 |
| 34.0 | 1.8 | 949 | 2.64 |
| 34.3 | 0.6 | 321 | 2.61 |
| 34.9 | 0.6 | 321 | 2.57 |
| 35.1 | 0.6 | 328 | 2.55 |
| 36.1 | 2 | 1057 | 2.48 |
| 36.5 | 0.9 | 456 | 2.46 |
| 37.1 | 0.6 | 318 | 2.42 |
| 37.7 | 0.5 | 264 | 2.38 |
| 38.3 | 0.5 | 271 | 2.35 |
| 38.6 | 0.8 | 391 | 2.33 |
| 38.8 | 1.1 | 551 | 2.32 |
| 39.4 | 0.8 | 409 | 2.29 |

### Free Base Form III

Free Base Form III was obtained by drying Form IV at 50° C. under vacuum. The XRPD peak list (±0.2 degrees 2Θ) of Free Base Form III is provided in Table B.

TABLE B

| Peak (2Θ °) | Intensity % | Intensity Count | d-spacing (Å) |
|---|---|---|---|
| 7.6 | 38.2 | 1471 | 11.64 |
| 9.4 | 21.2 | 816 | 9.45 |
| 14.8 | 57.5 | 2214 | 5.99 |
| 15.5 | 25.9 | 999 | 5.72 |
| 15.8 | 17.2 | 664 | 5.60 |
| 17.1 | 25.2 | 970 | 5.18 |
| 17.5 | 20.7 | 796 | 5.07 |
| 18.1 | 69 | 2655 | 4.91 |
| 18.7 | 14.2 | 545 | 4.73 |
| 19.2 | 15.6 | 600 | 4.62 |
| 19.9 | 100 | 3850 | 4.46 |
| 21.5 | 10.9 | 421 | 4.13 |
| 22.1 | 24.9 | 957 | 4.01 |
| 22.8 | 22 | 848 | 3.90 |
| 24.2 | 9.7 | 375 | 3.67 |
| 25.1 | 9.1 | 350 | 3.55 |
| 26.0 | 25.1 | 967 | 3.42 |
| 27.1 | 7.2 | 278 | 3.29 |
| 27.7 | 18.9 | 726 | 3.22 |
| 28.4 | 11.1 | 427 | 3.14 |
| 29.9 | 11 | 424 | 2.99 |
| 31.2 | 7.6 | 292 | 2.87 |
| 31.9 | 5.6 | 216 | 2.80 |
| 32.7 | 7.3 | 280 | 2.74 |
| 33.8 | 7.6 | 291 | 2.65 |
| 35.4 | 4.8 | 183 | 2.53 |
| 38.5 | 5.4 | 206 | 2.34 |
| 39.0 | 5.2 | 200 | 2.31 |
| 39.7 | 4.5 | 172 | 2.27 |

### Free Base Form IV

Free Base Form IV was obtained by slurrying Compound 1 in water at room temperature. The XRPD peak list (±0.2 degrees 2Θ) of Free Base Form IV is provided in Table C.

TABLE C

| Peak (2Θ °) | Intensity % | Intensity Count | d-spacing (Å) |
|---|---|---|---|
| 3.3 | 35.8 | 678 | 27.03 |
| 6.2 | 38.7 | 732 | 14.33 |
| 7.0 | 31.1 | 588 | 12.59 |
| 9.0 | 19.1 | 362 | 9.85 |
| 9.7 | 11.6 | 219 | 9.08 |
| 12.4 | 8.5 | 161 | 7.14 |
| 13.8 | 49.8 | 943 | 6.42 |
| 14.1 | 16.2 | 307 | 6.27 |
| 14.5 | 44.1 | 834 | 6.10 |
| 14.8 | 73.9 | 1399 | 5.98 |
| 15.9 | 21.2 | 402 | 5.56 |
| 16.4 | 15.9 | 300 | 5.40 |
| 18.1 | 100 | 1892 | 4.90 |
| 18.7 | 25.6 | 484 | 4.74 |
| 19.1 | 68.2 | 1291 | 4.65 |
| 19.3 | 27.2 | 514 | 4.58 |
| 19.9 | 59.4 | 1123 | 4.46 |
| 20.6 | 61.3 | 1160 | 4.32 |
| 21.7 | 19.5 | 368 | 4.10 |
| 22.4 | 21.1 | 399 | 3.96 |
| 22.9 | 12.1 | 229 | 3.88 |
| 23.6 | 35.1 | 665 | 3.77 |
| 24.5 | 43.3 | 819 | 3.63 |
| 25.1 | 10 | 189 | 3.55 |
| 25.5 | 23.9 | 452 | 3.49 |
| 26.2 | 37.8 | 716 | 3.40 |
| 26.6 | 34.1 | 646 | 3.35 |
| 27.3 | 11.6 | 220 | 3.27 |
| 27.9 | 11.5 | 218 | 3.19 |
| 28.8 | 20.4 | 386 | 3.10 |
| 29.3 | 8.8 | 167 | 3.04 |
| 29.8 | 19.6 | 370 | 3.00 |
| 30.4 | 10.3 | 195 | 2.93 |
| 30.9 | 14.1 | 266 | 2.89 |
| 31.7 | 9.2 | 174 | 2.82 |
| 32.2 | 9.7 | 183 | 2.78 |
| 32.6 | 8.2 | 156 | 2.74 |
| 33.1 | 7.2 | 136 | 2.70 |
| 34.2 | 6.1 | 116 | 2.62 |
| 35.9 | 6.1 | 116 | 2.50 |
| 39.3 | 8 | 151 | 2.29 |

### HCl Form I

HCl Form I was obtained by dissolving Compound 1 in acetonitrile and 3 M HCl in water, stirring at room temperature for 2 days with slow evaporation, and drying the crystals thus obtained vacuum at 50° C. for ˜ 4 hours. The XRPD peak list (=0.2 degrees 28) of HCl Form I is provided in Table D.

TABLE D

| Peak (2Θ °) | Intensity % | Intensity Count | d-spacing (Å) |
|---|---|---|---|
| 4.3 | 14.2 | 393 | 20.63 |
| 7.5 | 8.7 | 242 | 11.81 |
| 12.5 | 9.2 | 254 | 7.09 |
| 13.0 | 12.6 | 350 | 6.79 |
| 13.6 | 49.7 | 1378 | 6.52 |
| 14.1 | 15.4 | 428 | 6.28 |
| 14.6 | 71.2 | 1975 | 6.08 |
| 14.8 | 24 | 666 | 5.97 |
| 15.2 | 14.7 | 408 | 5.81 |
| 15.5 | 13.2 | 366 | 5.71 |
| 16.8 | 24.8 | 689 | 5.27 |
| 17.6 | 21 | 582 | 5.02 |
| 18.6 | 30.2 | 839 | 4.77 |
| 19.5 | 6.1 | 168 | 4.54 |
| 20.3 | 23.7 | 659 | 4.38 |
| 21.1 | 24.7 | 686 | 4.21 |
| 21.6 | 20.6 | 572 | 4.12 |
| 21.9 | 7.6 | 211 | 4.06 |
| 22.6 | 40.3 | 1117 | 3.94 |
| 23.2 | 4.9 | 137 | 3.83 |
| 23.9 | 16.5 | 457 | 3.72 |
| 24.1 | 50.9 | 1413 | 3.68 |
| 25.0 | 100 | 2775 | 3.56 |
| 25.4 | 22.8 | 632 | 3.51 |
| 25.8 | 18.2 | 505 | 3.45 |
| 26.4 | 51.2 | 1422 | 3.37 |
| 27.0 | 7 | 194 | 3.29 |
| 27.6 | 17.3 | 480 | 3.23 |
| 28.0 | 11 | 305 | 3.18 |
| 28.3 | 7.9 | 218 | 3.15 |
| 28.7 | 5.9 | 165 | 3.11 |
| 29.2 | 13.4 | 371 | 3.05 |
| 29.8 | 9.7 | 270 | 3.00 |
| 30.3 | 19.3 | 535 | 2.95 |
| 30.7 | 6.9 | 191 | 2.91 |
| 31.3 | 11.2 | 311 | 2.85 |
| 31.9 | 6.7 | 187 | 2.80 |
| 32.8 | 7.4 | 206 | 2.73 |
| 33.3 | 6.1 | 170 | 2.69 |
| 34.1 | 5.9 | 165 | 2.63 |
| 34.3 | 5.5 | 153 | 2.61 |
| 35.0 | 6.7 | 187 | 2.56 |
| 35.4 | 4.4 | 122 | 2.53 |
| 36.4 | 6.5 | 180 | 2.47 |
| 37.3 | 7.4 | 206 | 2.41 |
| 38.1 | 5 | 139 | 2.36 |
| 38.3 | 6.7 | 187 | 2.35 |
| 38.8 | 9.1 | 252 | 2.32 |
| 39.2 | 4.6 | 129 | 2.30 |

### HCl Form II

HCl Form I was obtained by dissolving HCl Form I in acetone and 1.1 eq. HCl solution in water at 50° C., cooling to 40° C. at 0.1° C./min, seeding the solution, and holding for 2 hours. The resulting suspension was cooled to 20° C. at 0.1° C./min and then stirred overnight/weekend. After filtration, the solid thus obtained was HCl Form II. The XRPD peak list (±0.2 degrees 2Θ) of HCl Form II is provided in Table E.

TABLE E

| Peak (2Θ °) | Intensity % | Intensity Count | d-spacing (Å) |
|---|---|---|---|
| 6.6 | 357 | 3.2% | 13.41 |
| 7.7 | 660 | 23.4% | 11.46 |
| 9.3 | 541 | 20.9% | 9.47 |
| 10.9 | 379 | 13.0% | 8.10 |
| 13.0 | 390 | 13.7% | 6.81 |
| 13.5 | 236 | 5.2% | 6.55 |
| 14.2 | 538 | 21.5% | 6.22 |
| 15.3 | 1474 | 71.5% | 5.80 |
| 16.0 | 1772 | 87.3% | 5.55 |
| 16.8 | 1109 | 51.3% | 5.27 |
| 17.7 | 2003 | 100.0% | 5.01 |
| 18.7 | 598 | 24.5% | 4.74 |
| 19.8 | 609 | 25.6% | 4.47 |
| 20.2 | 363 | 12.4% | 4.40 |
| 21.6 | 619 | 26.2% | 4.11 |
| 22.6 | 1645 | 82.0% | 3.93 |
| 23.1 | 410 | 15.2% | 3.85 |
| 23.8 | 253 | 7.2% | 3.73 |
| 25.2 | 172 | 2.9% | 3.53 |
| 25.9 | 303 | 9.5% | 3.44 |
| 26.2 | 443 | 17.0% | 3.40 |
| 26.5 | 255 | 6.7% | 3.36 |
| 27.4 | 636 | 27.4% | 3.25 |
| 27.9 | 1094 | 52.4% | 3.20 |
| 28.6 | 504 | 20.9% | 3.11 |
| 30.0 | 392 | 15.4% | 2.98 |
| 30.4 | 210 | 5.6% | 2.94 |
| 31.1 | 242 | 7.6% | 2.87 |
| 31.4 | 175 | 4.2% | 2.84 |
| 32.7 | 205 | 5.7% | 2.74 |
| 33.0 | 182 | 4.4% | 2.71 |
| 33.7 | 288 | 10.3% | 2.66 |
| 35.0 | 175 | 4.9% | 2.56 |
| 38.1 | 177 | 5.1% | 2.36 |
| 39.2 | 128 | 2.2% | 2.30 |
| 39.7 | 170 | 3.5% | 2.27 |

### HBr Form I

HBr Form I was obtained Compound 1 in acetonitrile and 1.1 eq. HBr solution in water. The solution was stirred at room temperature overnight and a solid precipitated. The solid was filtered and dried at 50° C. under vacuum to give HBr Form I. The XRPD peak list (=0.2 degrees 2Θ) of HBr Form I is provided in Table F.

TABLE F

| Angle 2Θ ° | Intensity % | Intensity Count | d-spacing Å |
|---|---|---|---|
| 3.2 | 38 | 642 | 27.48 |
| 7.6 | 33.1 | 560 | 11.67 |
| 12.3 | 23.7 | 400 | 7.20 |
| 12.9 | 32.3 | 546 | 6.88 |
| 13.6 | 17.4 | 294 | 6.50 |
| 14.1 | 26.6 | 450 | 6.27 |
| 14.4 | 24.7 | 417 | 6.13 |
| 15.0 | 47.4 | 801 | 5.88 |
| 15.6 | 19 | 322 | 5.69 |
| 16.7 | 17.3 | 292 | 5.29 |
| 17.7 | 45.1 | 762 | 5.01 |
| 18.8 | 89.8 | 1518 | 4.71 |
| 19.5 | 31.5 | 532 | 4.55 |
| 20.3 | 41.7 | 705 | 4.36 |
| 21.2 | 63.2 | 1069 | 4.19 |
| 22.6 | 70 | 1184 | 3.94 |
| 23.3 | 15.6 | 263 | 3.81 |
| 24.2 | 56.4 | 954 | 3.68 |
| 24.7 | 27.3 | 462 | 3.60 |
| 25.1 | 100 | 1691 | 3.55 |
| 25.9 | 28.9 | 488 | 3.44 |
| 26.1 | 33.4 | 564 | 3.42 |
| 26.5 | 82.3 | 1391 | 3.36 |
| 27.1 | 25.2 | 426 | 3.28 |
| 27.5 | 14 | 236 | 3.24 |
| 28.4 | 24.2 | 409 | 3.14 |
| 29.3 | 24.4 | 413 | 3.04 |
| 29.6 | 20.2 | 341 | 3.01 |
| 30.6 | 39.2 | 663 | 2.92 |
| 31.2 | 37.3 | 631 | 2.86 |
| 32.1 | 15 | 254 | 2.78 |
| 32.3 | 14.5 | 246 | 2.77 |
| 32.9 | 16.1 | 273 | 2.72 |
| 33.4 | 18.9 | 319 | 2.68 |
| 33.9 | 15.5 | 262 | 2.64 |
| 34.5 | 13.8 | 234 | 2.60 |
| 35.6 | 11.7 | 198 | 2.52 |
| 36.0 | 17.7 | 300 | 2.49 |
| 36.5 | 17.8 | 301 | 2.46 |
| 37.0 | 15.2 | 257 | 2.42 |
| 37.6 | 11.1 | 187 | 2.39 |
| 38.4 | 17.3 | 293 | 2.34 |
| 39.0 | 16.4 | 277 | 2.31 |
| 39.5 | 11.9 | 201 | 2.28 |

### Example 2

### Film Coated Tablet Preparation

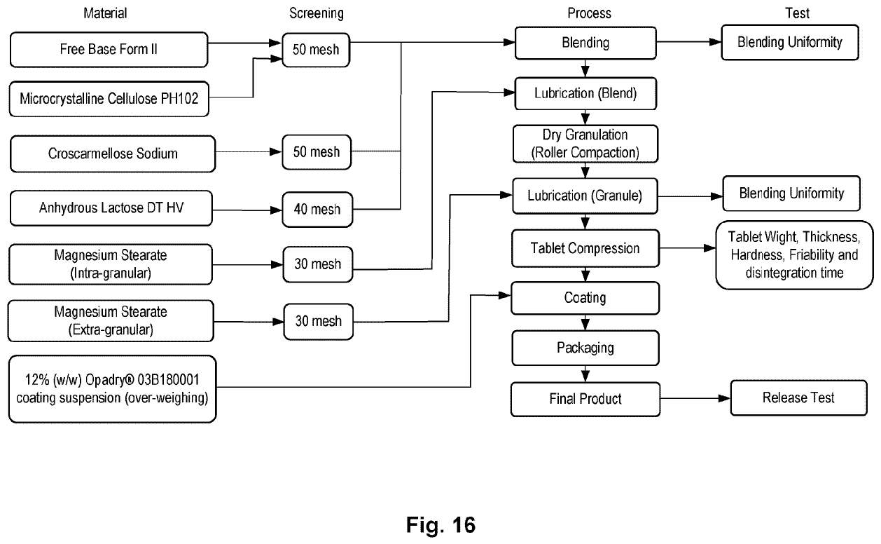

Table 2A shows the ingredients for film coated tables comprising 25 mg of Free Base Form II. The Process Flow Diagram is provided in FIG. 16.

TABLE 2A

Strength
25 mg
Process
Roller Compaction

| | | |
|---|---|---|
| Ingredient | mg/Tablet | w/w % |

Intra-granular

| | | |
|---|---|---|
| Free Base Form II | 25.00 | 25.00 |
| Microcrystalline Cellulose | 35.25 | 35.25 |
| PH102 | | |
| Anhydrous lactose DT HV | 35.25 | 35.25 |
| Croscarmellose Sodium | 3.00 | 3.00 |
| Magnesium Stearate | 0.50 | 0.50 |

Extra-granular

| | | |
|---|---|---|
| Magnesium Stearate | 1.00 | 1.00 |
| Total (core) | 100.00 | 100.00 |

Coating Material

| | | |
|---|---|---|
| Opadry ® 03B180001 | 3.00 | 3.00 |
| White*¹ | | |
| Purified Water*2 | N/A | N/A |
| Total (coated tablet) | 103.00 | N/A |

*¹Opadry ® 03B180001 White contains HPMC 2910/hypromellose, titanium dioxide and macrogol/PEG.
*2Purified water is removed during the coating process.

### Screening

Anhydrous lactose DT HV was passed through a 40 mesh (450 μm) screen. Free Base Form II was poured out of its bag onto a 50 mesh (355 μm) screen. The Free Base Form II bag was rinsed with microcrystalline cellulose PH102 and the Free Base Form II and microcrystalline cellulose PH102 co-screened through the 50 mesh screen.

Croscarmellose sodium was passed through the same 50 mesh screen into the same bag.

Magnesium stearate (intra-granular and extra-granular) were passed through a 30 mesh (600 μm) screen into separate bags.

### Blending and Lubrication (Blend)

The Free Base Form II and excipients, except for the intra-granular and extra-granular magnesium stearate, were charged into a 5 L blending bin and blended at 20 rpm for 20 minutes.

Six samples were taken from different blend locations at the selected time points, and tested for blend uniformity. The blending uniformity results are shown in Table 3A.

TABLE 3A

| | Blending | Blending Uniformity Results (%) | | | | | | | |
|---|---|---|---|---|---|---|---|---|---|
| Process | Time | 1 | 2 | 3 | 4 | 5 | 6 | Mean | RSD |
| Blending | 20 | 98.7 | 98.9 | 99.3 | 100.9 | 100.3 | 100.8 | 99.8 | 1.0 |

After blending, the screened 0.5% intra-granular magnesium stearate was charged into the blending bin and blending performed for 5 minutes at a speed of 20 rpm.

### Roller Compaction and Milling

The lubricated blend was passed through a roller compactor at different feed speeds to produce dry granules.

Sieve analysis results for the dry granules after roller compaction are shown in Table 4. Other granule properties are shown in Table 5.

TABLE 4
Feed Screw Speed (rpm) 22

| Mesh Size | Screen Hole | Amount |
|---|---|---|
| Range (Mesh) | Diameter (μm) | Retained (%) |
| >18 | >1000 | 0.10 |
| 18-40 | 425-1000 | 40.69 |
| 40-45 | 355-425 | 7.06 |
| 45-60 | 250-355 | 12.45 |
| 60-80 | 180-250 | 9.82 |
| 80-100 | 150-180 | 7.13 |
| 100-200 | 75-150 | 20.05 |
| <200 | <75 | 2.50 |

| | |
|---|---|
| Total | 99.80 |

TABLE 5

| Feed Screw Speed | Bulk Density | Tapped Density | Carr's Index |
|---|---|---|---|
| (rpm) | (g/mL) | (g/mL) | (%) |
| 22 | 0.599 | 0.824 | 27.3 |

### Lubrication (Granule)

1% extra-granular magnesium stearate was blended with the granule for 5 minutes. Six samples from different blend locations were taken and tested for blend uniformity. The blending uniformity results are shown in Table 6.

TABLE 6

| | Blending Uniformity Results (%) | | | | | | | |
|---|---|---|---|---|---|---|---|---|
| Process | 1 | 2 | 3 | 4 | 5 | 6 | Mean | RSD |
| Lubrication | 100.2 | 100.7 | 100.2 | 100.8 | 100.1 | 98.9 | 100.2 | 0.7 |
| (Granule) | | | | | | | | |

### Compression

The lubricated granules were compressed into tablets using a rotary compression machine. During compression, tablets were sampled at different stages, and tested for tablet weight, hardness and content uniformity.

### Coating

The tablet cores were coated with Opadry® Complete Film Coating System 03B180001 white. A coating suspension with a solids content of 12% was prepared. The coating weight gain was in the range of 3.0±0.5%.

The dissolution results for the resulting coated tablets are shown in Table 7.

TABLE 7

| | | | Dissolution (%) | | | | | |
|---|---|---|---|---|---|---|---|---|
| | Assay | Dissolution | 5 | 15 | 30 | 45 | 60 | 75 |
| Strength | (%) | Item | min | min | min | min | min | min |
| 25 mg | 100.3 | Mean | 45 | 94 | 97 | 98 | 98 | 98 |
| | | RSD % | 4.9 | 1.0 | 1.0 | 1.2 | 1.2 | 1.3 |

Content uniformity of the coated tablets was tested to further confirm the effect of coating process on tablet assay and the results are shown in Table 8.

TABLE 8

| No. | % Assay | % Average | S | AV | Standard |
|---|---|---|---|---|---|
| 1 | 100.5 | 101.0 | 0.972 | 2.3 | AV ≤ 15.0 |
| 2 | 102.0 | | | | |
| 3 | 102.6 | | | | |
| 4 | 100.9 | | | | |
| 5 | 102.2 | | | | |
| 6 | 100.4 | | | | |
| 7 | 99.7 | | | | |
| 8 | 100.8 | | | | |
| 9 | 100.3 | | | | |
| 10 | 100.2 | | | | |

The process parameters are summarized in Table 9.

TABLE 9

| Process Steps | Process Variables | Process Parameters | |
|---|---|---|---|
| Screening | Anhydrous lactose DT HV | 40 mesh screen | |
| | | (450 μm) | |
| | Free Base Form II | 50 mesh screen | |
| | Microcrystalline Cellulose | (355 μm) | |
| | PH102 | | |
| | Croscarmellose Sodium | | |
| | Magnesium Stearate | 30 mesh screen | |
| | | (600 μm) | |
| Blending | Bin Size | 5 | L |
| | Blending Speed × | 20 rpm × 2 0 min | |
| | Blending Time | | |
| Lubrication | Bin Size | 5 | L |
| (Blend) | Blending Speed × | 20 rpm × 5 min | |
| | Blending Time | | |
| Roller Compaction | Feed screw speed | 22 | rpm |
| and Milling | Roll pressure | 50 | bar |
| | Roll speed | 10 | rpm |
| | Mill speed | 100 | rpm |
| | Mill screen orifice size | 0.8 | mm |
| Lubrication | Bin Size | 2 | L |
| (Granule) | Blending Speed × | 20 rpm × 5 min | |
| | Blending Time | | |
| Subpackage | Lubricated granule | Material scoop | |
| Compression | Size of Compression | 6 mm round concave | |
| | Tooling | punches | |
| | Number of punches | 3 sets of punches | |
| | Feeding type | Gravity feed frame | |
| | Individual Tablet Core | 100 | mg |
| | Weight | | |
| | Hardness | 65N | |
| | Friability | ≤1.0% | |
| | Disintegration Time | ≤15 | min |
| Coating | Drum Diameter | 215 | mm |
| | Inlet Temperature | 50-80° | C. |
| | Drum Speed | 5-10 | rpm |
| | Air Flow Rate | 1000-2500 | rpm |
| | Atomization Pressure | 0.1-0.4 | MPa |
| | Pump Speed | 1-10 | rpm |
| | Product Temperature | 35-45° | C. |

| | |
|---|---|
| Weight Gain | 3.0% |

### Example 3

### Pharmacokinetics of Free Base Forms I and II

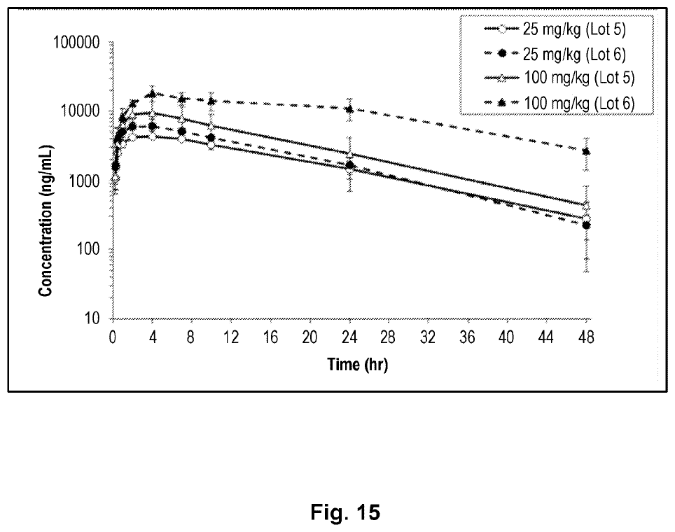

In this study, male Beagle dogs were administered Free Base Form I and Free Base Form II at 25 mg/kg and 100 mg/kg (0.5% carboxymethylcellulose (CMC) and 0.1% Tween in water at pH 7). The dogs were fasted overnight and then offered food one hour prior to oral administration of the form. Blood samples were collected at predetermined timepoints up to 48 hours. Compound 1 plasma concentrations were determined by liquid chromatography with tandem mass spectrometry (LC-MS/MS) methods. The concentration-time profiles are shown in FIG. 15 and the relevant PK parameters are summarized in Table 10.

TABLE 10

| | Free Base Form I | | | | Free Base Form II | | | |
|---|---|---|---|---|---|---|---|---|
| PK Parameters | 25 mg/kg^{a} | | 100 mg/kg^{a} | | 25 mg/kg^{a} | | 100 mg/kg^{a} | |
| Tₘₐₓ (h)^{b} | 2.0 | | 4.0 | | 4.0 | | 4.0 | |
| Cₘₐₓ (ng/mL) | 4290 | (6.5) | 10600 | (35.2) | 6170 | (24.6) | 18500 | (25.2) |
| AUC_{0-inf} (ng · h/mL) | 90600 | (22.5) | 166000 | (53.1) | 109000 | (9.6) | 520000 | (33.9) |

Abbreviations: AUC_{0-inf} = area under the plasma concentration-time curve from time 0 extrapolated to infinity; Cₘₐₓ = maximum plasma concentration; CV = coefficient of variation; PK = pharmacokinetics; Tₘₐₓ = time of peak plasma concentration; h = hours postdose.
^{a}n = 3/group
^{b}Tmax = median values.

The results demonstrate that Free Base Form II gives an unexpectedly greater exposure in dogs than did Free Base Form I. This difference was dose-dependent. At the 25 mg/kg dose, the Compound 1 Cₘₐₓ and area under the plasma concentration-time curve from time 0 extrapolated to infinity (AUC0-inf) of Free Base Form II was ˜40% and 20% greater, respectively, than those of Free Base Form I. The Compound 1 exposure difference was amplified at the 100 mg/kg dose with the Cₘₐₓ and AUC_{0-inf} of Free Base Form II being 1.8-fold and 3.1-fold greater, respectively, than those of Free Base Form I. The result suggests that Free Base Form II was more extensively absorbed than Free Base Form I, especially at the dose of 100 mg/kg. Without wishing to be bound by any particular theory, the more extensive absorption of Free Base Form II may be due to disparities in solubility or dissolution process of these forms in the dog intestinal lumen.

It is to be understood that the foregoing described embodiments and exemplifications are not intended to be limiting in any respect to the scope of the disclosure, and that the claims presented herein are intended to encompass all embodiments and exemplifications whether or not explicitly presented herein.

All patents and publications cited herein are fully incorporated by reference in their entirety.

What is claimed is:

1. A crystalline form of:
(a) N-((1R,3S)-3-(4-acetylpiperazin-1-yl)cyclohexyl)-4-fluoro-7-methyl-1H-indole-2-carboxamide, having Free Base Form II, Free Base Form III, or Free Base Form IV, or a mixture thereof; or
(b) N-((1R,3S)-3-(4-acetylpiperazin-1-yl)cyclohexyl)-4-fluoro-7-methyl-1H-indole-2-carboxamide hydrochloride, having HCl Form I or HCl Form II, or a mixture thereof; or
(c) N-((1R,3S)-3-(4-acetylpiperazin-1-yl)cyclohexyl)-4-fluoro-7-methyl-1H-indole-2-carboxamide hydrobromide, having HBr Form I,
wherein:
(i) Free Base Form II is characterized as having a powder x-ray diffraction pattern with peaks at 7.0, 14.0, 18.0, and 20.2 degrees 2Θ using Cu Kα radiation, wherein the 2Θ values are ±0.2 degrees 2Θ;
(ii) Free Base Form III is characterized as having a powder x-ray diffraction pattern with peaks at 7.6, 14.8, 18.0, and 19.8 degrees 2Θ using Cu Kα radiation, wherein the 2Θ values are ±0.2 degrees 2Θ;
(iii) Free Base Form IV is characterized as having a powder x-ray diffraction pattern with peaks at 14.8, 18.1, 19.1, 19.9, and 20.5 degrees 2Θ using Cu Kα radiation, wherein the 2Θ values are ±0.2 degrees 2Θ;
(iv) HCl Form I is characterized as having a powder x-ray diffraction pattern with peaks at 14.6 and 25.0 degrees 2Θ using Cu Kα radiation, wherein the 2Θ values are ±0.2 degrees 2Θ;
(v) HCl Form II is characterized as having a powder x-ray diffraction pattern with peaks at 15.2, 16.0, 17.7, and 22.6 degrees 2Θ using Cu Kα radiation, wherein the 2Θ values are ±0.2 degrees 2Θ; and
(vi) HBr Form I is characterized as having a powder x-ray diffraction pattern with peaks at 18.8, 25.1, and 26.5 degrees 2Θ using Cu Kα radiation, wherein the 2Θ values are ±0.2 degrees 2Θ.

2. The crystalline form of claim 1 which is N-((1R,3S)-3-(4-acetylpiperazin-1-yl)cyclohexyl)-4-fluoro-7-methyl-1H-indole-2-carboxamide having Free Base Form II.

3. The crystalline form of claim 1 which is N-((1R,3S)-3-(4-acetylpiperazin-1-yl)cyclohexyl)-4-fluoro-7-methyl-1H-indole-2-carboxamide having Free Base Form III.

4. The crystalline form of claim 1 which is N-((1R,3S)-3-(4-acetylpiperazin-1-yl)cyclohexyl)-4-fluoro-7-methyl-1H-indole-2-carboxamide having Free Base Form IV.

5. The crystalline form of any one of claims 2-4 comprising about 5% or less of any other physical form of N-((1R,3S)-3-(4-acetylpiperazin-1-yl)cyclohexyl)-4-fluoro-7-methyl-1H-indole-2-carboxamide.

6. The crystalline form of claim 1 which is N-((1R,3S)-3-(4-acetylpiperazin-1-yl)cyclohexyl)-4-fluoro-7-methyl-1H-indole-2-carboxamide hydrochloride having HCl Form I.

7. The crystalline form of claim 1 which is N-((1R,3S)-3-(4-acetylpiperazin-1-yl)cyclohexyl)-4-fluoro-7-methyl-1H-indole-2-carboxamide hydrochloride having HCl Form II.

8. The crystalline form of claim 6 or 7 comprising about 5% or less of any other physical forms of N-((1R,3S)-3-(4-acetylpiperazin-1-yl)cyclohexyl)-4-fluoro-7-methyl-1H-indole-2-carboxamide hydrochloride.

9. The crystalline form of claim 1 which is N-((1R,3S)-3-(4-acetylpiperazin-1-yl)cyclohexyl)-4-fluoro-7-methyl-1H-indole-2-carboxamide hydrochloride having HBr Form I.

10. The crystalline form of claim 9 comprising about 5% or less of any other physical forms of N-((1R,3S)-3-(4-acetylpiperazin-1-yl)cyclohexyl)-4-fluoro-7-methyl-1H-indole-2-carboxamide hydrobromide.

11. A pharmaceutical composition comprising the crystalline form of any one of claims 1-10 and one or more pharmaceutically acceptable excipients.

12. The pharmaceutical composition of claim 11, wherein the one or more pharmaceutically acceptable excipients comprise a ductile diluent, a brittle diluent, a disintegrant, a binder, a glidant, or a lubricant, or a combination thereof.

13. The pharmaceutical composition of claim 11, wherein the one or more pharmaceutically acceptable excipients comprise microcrystalline cellulose, partially pregelatinized maize starch, anhydrous lactose, mannitol, dicalcium phosphate anhydrous, croscarmellose sodium, sodium starch glycolate, crospovidone, hydroxypropyl cellulose, colloidal silicon dioxide, magnesium stearate, or stearic acid, or a combination thereof.

14. The pharmaceutical composition of claim 13, comprising:
(a) about 25% w/w of the crystalline form;
(b) about 35% w/w of microcrystalline cellulose;
(c) about 35% w/w of anhydrous lactose;
(d) about 3% w/w of croscarmellose sodium; and
(e) about 1.5% w/w of magnesium stearate.

15. The pharmaceutical composition of claim 14 further comprising a film coating.

16. The pharmaceutical composition of claim 15, wherein the film coating composition comprises HPMC 2910/hypromellose, titanium dioxide, and macrogol/PEG.

17. The pharmaceutical composition of any one of claims 11-16 formulated as a film coated tablet.

18. A method of making the pharmaceutical composition of any one of claims 11-17, the method comprising blending the crystalline form with the one or more pharmaceutically acceptable excipients.

19. A method of treating cancer in a subject in need thereof, the method comprising administering a therapeutically effective amount of the crystalline form of any one of claims 1-10, or a therapeutically effective amount of the pharmaceutical composition of any one of claims 11-17 to the subject.

20. The method of claim 19, wherein the cancer is any one or more of the cancers of Table 2.

21. The method of claim 19, wherein the cancer is a hematological cancer.

22. The method of claim 21, wherein the hematological cancer is any one or more of the cancers of Table 3.

23. The method of claim 22, wherein the hematological cancer is diffuse large B-cell lymphoma.

24. The method of claim 22, wherein the hematological cancer is mantle cell lymphoma.

25. The method of claim 22, wherein the hematological cancer is multiple myeloma.

26. The method of claim 25, wherein the hematological cancer is t(4; 14) multiple myeloma.

27. The method of any one of claims 19-26 further comprising administering a therapeutically effective amount of an anti-cancer agent to the subject.

28. The method of claim 27, where the anti-cancer agent comprises one or more glucocorticoid receptor agonists, one or more immunomodulatory drugs, one or more proteasome inhibitors, one or more Bcl-2 inhibitors, one or more pleiotropic pathway modulators, one or more XPO1 inhibitors, one or more histone deacetylase inhibitors, one or more EZH2 inhibitors, one or more BTK inhibitors, one or more anti-CD20 monoclonal antibodies, one or more alkylating agents, one or more topoisomerase II inhibitors, one or more vinca alkaloids, one or more platinum-based drugs, one or more nucleoside anticancer agents, one or more PI3K inhibitors, one or more CDK4/6 inhibitors, or one or more CARM1 inhibitors, or a combination thereof.

29. The method claim 27 or 28, wherein the anti-cancer agent comprises a glucocorticoid receptor agonist.

30. The method of claim 29, wherein the glucocorticoid receptor agonist is dexamethasone.

31. The method of any one of claims 27-30, wherein the anti-cancer agent comprises an immunomodulatory drug.

32. The method of claim 31, wherein the immunomodulatory drug is pomalidomide or lenalidomide.

33. The method of any one of claims 27-32, wherein the anti-cancer Agent comprises a proteasome inhibitor.

34. The method of claim 33, wherein the proteasome inhibitor is bortezomib.

35. The method of any one of claims 27-34, wherein the anti-cancer agent comprises a Bcl-2 inhibitor.

36. The method of claim 35, wherein the Bcl-2 inhibitor is venetoclax.

37. The method of any one of claims 27-36, wherein the anti-cancer agent comprises a pleiotropic pathway modulator.

38. The method of claim 37, wherein the pleiotropic pathway modulator is CC-122.

39. The method of any one of claims 27-38, wherein the anti-cancer agent comprises a XPO1 inhibitor.

40. The method of claim 39, wherein the XPO1 inhibitor is selinexor.

41. The method of any one of claims 27-40, wherein the anti-cancer agent comprises a histone deacetylase inhibitor.

42. The method of claim 41, wherein the histone deacetylase inhibitor is panobinostat.

43. The method of any one of claims 27-42, wherein the anti-cancer agent is an EZH2 inhibitor.

44. The method of claim 43, wherein the EZH2 inhibitor is tazemetostat.

45. The method of any one of claims 27-44, wherein the anti-cancer agent comprises a BTK inhibitor.

46. The method of claim 45, wherein the BTK inhibitor is ibrutinib, acalabrutinib, or zanubrutinib.

47. The method of any one of claims 27-46, wherein the anti-cancer agent comprises an anti-CD20 monoclonal antibody.

48. The method of claim 47, wherein the anti CD20 monoclonal antibody is rituximab.

49. The method of any one of claims 27-48, wherein the anti-cancer agent comprises a PI3K inhibitor.

50. The method of claim 49, wherein the PI3K inhibitor is copanlisib.

51. The method of any one of claims 27-50, wherein the anti-cancer agent comprises a CDK4/6 inhibitor.

52. The method of claim 51, wherein the CDK4/6 inhibitor is palbociclib.

53. The method of any one of claims 27-52, wherein the anti-cancer agent comprises a CARM1 inhibitor.

54. The method of claim 53, wherein the CARM1 inhibitor is EZM2302.

55. The method of any one of claims 27-54, wherein the anti-cancer agent comprises an alkylating agent.

56. The method of claim 55, wherein the alkylating agent is mafosfamide.

57. The method of any one of claims 27-56, wherein the anti-cancer agent comprises a topoisomerase II inhibitor.

58. The method of claim 57, wherein the topoisomerase II inhibitor is doxorubicin and etoposide.

59. The method of any one of claims 27-58, wherein the anti-cancer agent comprises a vinca alkaloid.

60. The method of claim 59, wherein the vinca alkaloid is vincristine.

61. The method of any one of claims 27-60, wherein the anti-cancer agent comprises a platinum-based drug.

62. The method of claim 61, wherein the platinum-based drug is carboplatin or oxaliplatin.

63. The method of any one of claims 27-62, wherein the anti-cancer agent comprises a nucleoside anticancer agent.

64. The method of claim 63, wherein the nucleoside anticancer agent is gemcitabine.

65. A kit comprising the crystalline form of any one of claims 1-10, or the pharmaceutical composition of any one of claims 11-17, and instructions for administering the crystalline form or the pharmaceutical composition to a subject in need thereof.

66. The kit of claim 65 further comprising an anti-cancer agent.

67. A kit for carrying out the method of any one of claims 19-26, the kit comprising (a) the crystalline form or the pharmaceutical composition; and (b) instructions for administering the crystalline form or the pharmaceutical composition the subject.

68. A kit for carrying out the method of any one of claims 27-64, the kit comprising (a) the crystalline form or the pharmaceutical composition; (b) instructions for administering the crystalline form or the pharmaceutical composition to the subject; (c) the anti-cancer agent; and (d) instructions for administering the anti-cancer agent to the subject.

69. A method of making the Free Base Form II of claim 1, the method comprising:
(i) heating N-((1R,3S)-3-(4-acetylpiperazin-1-yl)cyclohexyl)-4-fluoro-7-methyl-1H-indole-2-carboxamide in ethanol and water to give a solution;
(ii) cooling the solution to about 0° C.;
(iii) optionally seeding the solution; and
(iv) isolating the Free Base Form II.
